(19) United States
(12) Patent Application Publication
(10) Pub. No.: US 2023/0223131 A1
(43) Pub. Date: Jul. 13, 2023

(54) SYSTEMS AND METHODS FOR PROVIDING INDIVIDUALIZED RECOMMENDATIONS FOR A HEALTHY MICROBIOME

(71) Applicant: SOCIETE DES PRODUITS NESTLE S.A., Vevey (CH)

(72) Inventors: CHIEH JASON CHOU, Chexbres (CH); PALOMA ELORTEGUI PASCUAL, Epalinges (CH); NASHMIL EMAMI, Morges (CH); ROKO PLESTINA, Morges (CH); ERIC SCUCCIMARRA, Lausanne (CH); DANIELA PROZOROVSCAIA, Paudex (CH); SARAH NICOLE QUICK, Oberageri (CH)

(21) Appl. No.: 17/996,948

(22) PCT Filed: Apr. 26, 2021
(86) PCT No.: PCT/EP2021/060788

§ 371 Date: Oct. 24, 2022

(30) Foreign Application Priority Data
Apr. 27, 2020 (EP) 20171451.6

(51) Int. Cl. G16H 20/60; G16H 10/60

(52) U.S. Cl. CPC G16H 20/60; G16H 10/60

(57) ABSTRACT

The present invention relates to systems and methods for providing individualized microbiome recommendations to improve or maintain microbiome health. In several embodiments of the invention, the individualized microbiome health recommendations are diets, menus and recipes for improving or maintaining a healthy microbiome. In several embodiments, the microbiome-healthy recommendations are delivered by a computer-implemented system.

### FIELD OF THE INVENTION

The present invention relates to systems and methods for providing individualized microbiome recommendations to improve or maintain microbiome health. In several embodiments of the invention, the individualized microbiome health recommendations are diets, menus and recipes for improving or maintaining a healthy microbiome. In several embodiments, the microbiome-healthy recommendations are delivered by a computer-implemented system.

### BACKGROUND TO THE INVENTION

The gut microbiota is host to trillions of microorganisms living in the intestine, with majority hosted in the colon. Alterations in the composition and functions of gut microbiota are associated with many diseases and conditions such as metabolic and inflammatory disorders, cancer, depression, as well as, infant health and longevity.

Although numerous factors may affect the gut microbiota throughout one's lifespan, diet is considered to be amongst the most important. As no two microbiomes are the same between individuals, there is a need for methods and systems to provide individualized and personalized recommendations for microbiome health. In particular, such recommendations should be in a user-friendly manner for individuals to follow in order to improve or maintain their microbiome health.

### SUMMARY OF THE INVENTION

Individual food item recommendations for a healthy microbiome are often difficult for individual users to implement as they are not considered in the context of an entire diet, menus, or recipes over different meals throughout a day or over several weeks.

The methods and systems of the present invention advantageously implement clinically proven food recommendations into user-friendly, practical, and actionable microbiome-healthy diet, menu plans or recipes that can be adapted to specific individual needs and preferences. In this way, the individual user is provided clear guidance on how to implement microbiome-healthy recommendations in their daily diet, menus and recipes.

In several embodiments, the present invention advantageously determines that for the microbiome-healthy recommendations, the recommended daily allowance for an overall healthy diet are maintained. In particular, the requirements for micronutrients are observed, for example, the micronutrient requirements for daily vitamins and minerals.

A further advantage of several embodiments of the present invention is that for the microbiome-healthy recommendations, individual user dietary preferences such as: gluten-free, lactose-free, Mediterranean, vegan or vegetarian diets have been implemented when constructing the microbiome-healthy menu plans.

Another advantage of several embodiments of the invention is that total energy requirements for daily diets can be set at different thresholds for individuals who have different physical activity levels or wish to reduce their daily energy consumption in order to lose weight while still maintaining the microbiome-healthy recommendations.

### DESCRIPTION OF FIGURES

FIG. 1—Computer implemented system for microbiome-healthy recommendations: a block diagram of an example system according to one embodiment of the present disclosure

FIG. 2—Example of a Recommendation System for Healthy Microbiome

FIG. 3—Protein context per diet type for microbiome healthy menu plans

FIG. 4—Carbohydrate per diet type for microbiome healthy menu plans

FIG. 5—Total fat per diet type for microbiome healthy menu plans

FIG. 6—Vitamin K per diet type for microbiome healthy menu plans

FIG. 7—Food Folate per diet type for microbiome healthy menu plans

FIG. 8—Sodium per diet type for microbiome healthy menu plans

FIG. 9—Fiber per diet type for microbiome healthy menu plans

FIG. 10—Workflow optimization for building the microbiome healthy menu planner

FIG. 11—Individual biometric data for a typical user

FIG. 12—Sample microbiome-healthy menu plan for one day

FIG. 12A shows the nutritional content of a menu plan for one day.

FIG. 12B shows breakfast and lunch suggestions.

FIG. 12C shows dinner and snack suggestions. Meal or food item selections can be marked with a tag to show which selections are microbiome-healthy. In the snack selection, there is the possibility to select alternative items to substitute. In this example, the pistachios can be selected or alternatively pistachios roasted without salt may be selected. The images were marked with a small “bacterium symbol” 1202 for those tagged as microbiome-healthy, as they contained one of the microbiome-healthy rules. The “arrows symbol” 1204 allowed the user to interchange the recipes or dishes created automatically by the engine. The meal nutritional score, marked with a symbol, “My Menu IQ” 1206, was displayed to give the meal nutritional score out of 100 for each meal occasion.

FIG. 13—Sample microbiome-healthy recipe

FIG. 13A describes the ingredients and the amounts in the recipe.

FIG. 13B describes the instructions how to make the recipe.

### DETAILED DESCRIPTION OF THE INVENTION

Definitions “Microbiome-health” can be evaluated by a number of different measurements including:

(i) determining the alpha diversity of microbial species in the intestine,

(ii) butyrate producing bacteria in the intestine, and

(iii) short chain fatty acid production in the intestine.

“Alpha diversity of microbial species in the intestine” summarizes the structure of an ecological community with respect to its richness (number of taxonomic groups), evenness (distribution of abundances of the groups), or both. In microbial ecology in the intestine, analyzing the alpha diversity of amplicon sequencing data is a common first approach to assessing differences between environments. A reduction in alpha diversity of microbial species in the intestine typically occurs over the lifespan with ageing individuals. In general, improving or maintaining the alpha diversity of microbial species in the intestine is an indication of a healthy microbiome.

“Butyrate producing bacteria in the intestine” is an important group of bacteria for a healthy microbiome. Members of the Firmicutes phylum, a classification of bacteria, are particularly known for their butyrate-producing capacities. This group of bacteria is responsible for produce butyrate through a natural fermentation process, and the resulting butyrate plays a crucial role in maintaining the homeostasis of host metabolism and gut microbiome diversity. A reduction in butyrate concentration and the butyrate producing bacteria in the gut are related to the development of a number of different diseases. In addition, consuming prebiotics such as vegetables, pulses, fruit and wholegrains may increase butyrate production in the gut. High-protein, high-fat, low-carbohydrate diets have also been shown to disrupt butyrate production in the microbiome. In general, improving or maintaining the butyrate concentration in the intestine is an indication of a healthy microbiome.

“Short chain fatty acid production in the intestine” is another important component for a healthy microbiome. Short-chain fatty acids (SCFAs) are fatty acids with fewer than 6 carbon (C) atoms and they are the main metabolites produced by the microbiota in the large intestine through the anaerobic fermentation of indigestible polysaccharides such as dietary fiber and resistant starch. SCFAs might influence gut-brain communication and brain function directly or indirectly. Wholegrains left intact appear to lead to higher production of short-chain fatty acids. In general, improving or maintaining the short chain fatty acid production in the intestine is an indication of a healthy microbiome.

In several embodiments, the systems and methods of the invention contribute to microbiome-health by providing microbiome-healthy recommendations such as diet recommendations, menu recommendations and recipe recommendations to improve or maintain the alpha diversity of microbial species in the intestine; improve or maintain the butyrate production in the intestine; and improve or maintain short chain fatty acid production in the intestine.

Various embodiments of the disclosed system satisfy the general goal, given a particular diet, to recommend a set of foods or menus or recipes in order to maintain or improve the overall microbiome health of the individual. The microbiome health depends on the general characteristics of the individual (gender, age, weight, body measurements, physical activity level, and other health-related conditions like pregnancy or lactation etc.) and the recommendations to maintain or improve the microbiome-health likewise depend on characteristics of the individual.

Microbiome health improvements or maintenance of microbiome health can be determined from a fecal sample taken from the subject, before and after the dietary recommendations of the present invention, by measurement of parameters such as: (i) alpha diversity of microbial species in the intestine, (ii) butyrate producing bacteria and (iii) short chain fatty acid production in the intestine. Thus, it can be determined over time, the microbiome-healthy improvements after the individual has followed the microbiome-healthy, diet, menu and recipe recommendations of the present invention.

In various embodiments, the system disclosed herein calculates and displays recommendations of food items, menus or recipes indicating the nutritional impact for the microbiome. In these embodiments, the system determines and stores one or more indications of the needs of the individual for whom the recommendations are being calculated, for an individual over a given period of time such as a meal, an entire day, a week or a month.

The system then enables the user to indicate consumables (such as food items) that he or she has consumed or plans to consume. For each indicated food item, a database or data store of the disclosed system stores an indication of the nutrient content, particularly the micronutrient content, per amount of that food item or menu or recipe. The system uses the nutritional content information, multiplied by the amount of food item consumed over time, to determine the total nutritional intake over a time period for that particular food item or menu or recipe.

In various embodiments, the disclosed system provides a recommendation function, wherein the system suggests combinations of foods or menus or recipes that will result in an improved or optimal microbiome-healthy menu. For example, if a user accesses the system after breakfast and indicates the foods he or she had for breakfast, the disclosed system may calculate a score for the breakfast foods, but may also determine what nutrients would need to be consumed over the remainder of the day, as well as how much energy to consume over the remainder of the day, for the individual to consume nutrients and energy in the optimal ranges for that day to have an overall healthy microbiome. In this embodiment, the system uses these calculated nutrient amounts to determine combinations of food that can be consumed throughout the remainder of the day to ensure that the individual's nutritional goals are achieved as fully as possible while still consuming a number of calories within that individual's optimal caloric intake range. Thus, the system disclosed herein can operate not only as a tracking system, but also as a recommendation engine to recommend consumables to help individuals reach their nutritional goal of a healthy microbiome.

The term “nutrient” is used repeatedly herein. In some embodiments, the term “nutrient” as used herein refers to compounds having a beneficial effect on the body e.g. to provide energy, growth or health. The term includes organic and inorganic compounds. As used herein the term nutrient may include, for example, macronutrients, micronutrients, essential nutrients, conditionally essential nutrients and phytonutrients. These terms are not necessarily mutually exclusive. For example, certain nutrients may be defined as either a macronutrient or a micronutrient depending on the particular classification system or list.

In various embodiments, the term “macronutrient” is used herein consistent with its well understood usage in the art, which generally encompasses nutrients required in large amounts for the normal growth and development of an organism. Macronutrients in these embodiments may include, but are not limited to, carbohydrates, fats, proteins, amino acids and water. Certain minerals may also be classified as macronutrients, such as calcium, chloride, or sodium.

In various embodiments, the term “micronutrient” is used herein consistent with its well understood usage in the art, which generally encompasses compounds having a beneficial effect on the body, e.g. to help provide energy, growth or health, but which are required in only minor or trace amounts. The term in such embodiments may include or encompass both organic and inorganic compounds, e.g. individual amino acids, nucleotides and fatty acids; vitamins, antioxidants, minerals, trace elements, e.g. iodine, and electrolytes, e.g. sodium, and salts thereof, including sodium chloride.

In several embodiments, micronutrients are calculated, in particular vitamins and minerals, for a particular food, menu, or recipe to determine microbiome healthy items. The system may tag such items as “microbiome-healthy” with an icon in order that they are readily identifiable to the user.

In various embodiments, food groups have been identified which are considered particularly microbiome-healthy. These food or nutrient groups are to be selected from the group consisting of:

(i) wholegrain foods;

(ii) beans and legumes;

(iii) fiber;

(iv) nuts and seeds; and

(v) omega-3 fatty acids.

In several embodiments, menus and recipes are selected based on these food or nutrient groups per time period in order to obtain a microbiome-healthy menu or recipe. The system allows for substitution of food items, menus and recipes in order to build meals throughout the day.

In several embodiments, the menus and recipes take into account the amount of the food or nutrient groups needed in order to have a balanced diet overall as well as being microbiome healthy.

In one embodiment, the food or nutrient groups are recommended in the following amounts:

(i) wholegrain foods in the total amount of about 31 to 477 g/day;

(ii) beans and legumes in the total amount of about 35 to 472 g/day

(iii) fiber in the total amount of about 16 to 95 g/day;

(iv) nuts and seeds in the total amount of about 6 to 192 g/day; and

(v) omega-3 fatty acids in the total amount of up to about 5200 mg/day.

In various embodiments, the system takes into account individual user preferences. Individual dietary preferences such as: gluten-free, lactose-free, Mediterranean diet, vegan diet, vegetarian diet and other specific diets.

Individual user likes or dislikes can be stored within the system so that some recommendations of food items, menus and recipes are avoided. The system may also store the frequency of such food items, menus and recipes so they can be varied in order to avoid boredom from the user in having the same menu or recipe each day.

In another embodiment, one or more devices carried by the user could provide real-time information to the system when the user is in a food purchasing establishment such as a grocery store or a restaurant. Devices such as RFID readers, NFC readers, wearable camera devices, and mobile phones could receive or determine (such as by scanning RFID tags, reading bar codes, or determining the physical location of a user) foods that are available to a user at a particular grocery store or restaurant. The disclosed system could then make microbiome-healthy recommendations taking into account what foods could be immediately purchased or consumed by the user.

In one such embodiment, when a user sits down at a restaurant, the disclosed system may push information to the user's mobile phone recommending that the user select certain items from the menu to optimize the user's individual microbiome-healthy menu for a given time period. In still other embodiments, a voice recognition feature recognizes inputs provided vocally by a user. In one such embodiment, the voice recognition system listens as a user orders at a restaurant; in other embodiments, the voice recognition system enables the user to speak directly the items he or she has consumed or will consume. In another embodiment, the disclosed system could use geolocation to provide appropriate exercise recommendations based on the user's location. For example, an app on a user's phone, tablet, or computer could provide the user (e.g., in a chat box) different activity tips if the user is at work, in a gym, or at home.

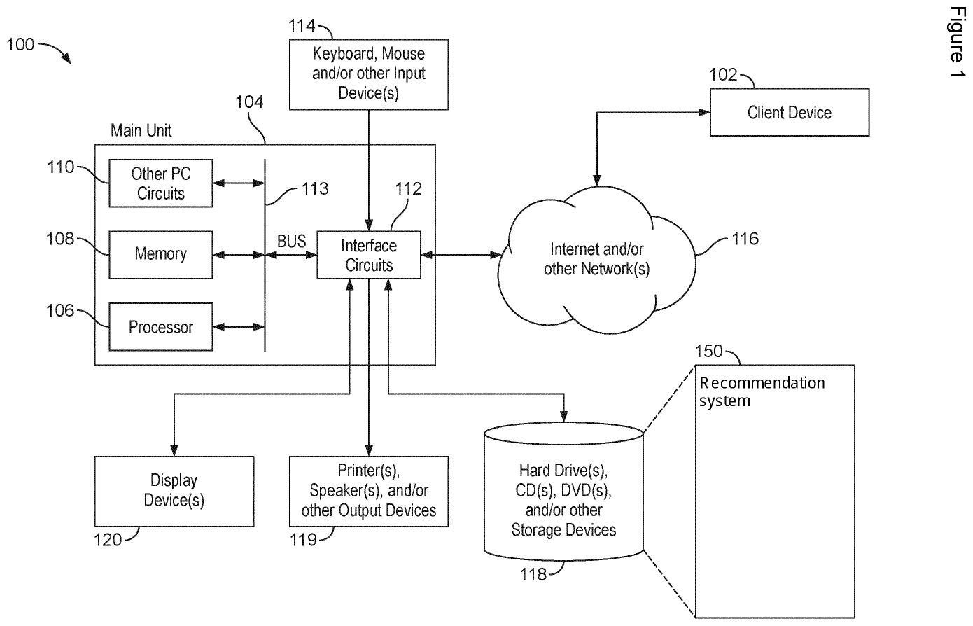

Referring now to FIG. 1, a block diagram is illustrated showing an example of the electrical systems of a host device 100 usable to implement at least portions of the computerized recommendation system disclosed herein.

In one embodiment, the device 100 illustrated in FIG. 1 corresponds to one or more servers and/or other computing devices that provide some or all of the following functions: (a) enabling access to the disclosed system by remote users of the system; (b) serving web page(s) that enable remote users to interface with the disclosed system; (c) storing and/or calculating underlying data, such as recommended caloric intake ranges, recommended nutrient consumption ranges, and nutrient content of foods, needed to implement the disclosed system; (d) calculating and displaying component; and/or (e) making recommendations of foods, menus or recipes or other consumables that can be consumed to help individuals reach an optimal healthy microbiome.

In the example architecture illustrated in FIG. 1, the device 100 includes a main unit 104 which preferably includes one or more processors 106 electrically coupled by an address/data bus 113 to one or more memory devices 108, other computer circuitry 110, and/or one or more interface circuits 112. The one or more processors 106 may be any suitable processor, such as a microprocessor from the INTEL PENTIUM® or INTEL CELERON® family of microprocessors. PENTIUM® and CELERON® are trademarks registered to Intel Corporation and refer to commercially available microprocessors. It should be appreciated that in other embodiments, other commercially-available or specially-designed microprocessors may be used as processor 106. In one embodiment, processor 106 is a system on a chip (“SOC”) designed specifically for use in the disclosed system.

In one embodiment, device 100 further includes memory 108. Memory 108 preferably includes volatile memory and non-volatile memory. Preferably, the memory 108 stores one or more software programs that interact with the hardware of the host device 100 and with the other devices in the system as described below. In addition or alternatively, the programs stored in memory 108 may interact with one or more client devices such as client device 102 (discussed in detail below) to provide those devices with access to media content stored on the device 100. The programs stored in memory 108 may be executed by the processor 106 in any suitable manner.

The interface circuit(s) 112 may be implemented using any suitable interface standard, such as an Ethernet interface and/or a Universal Serial Bus (USB) interface. One or more input devices 114 may be connected to the interface circuit 112 for entering data and commands into the main unit 104. For example, the input device 114 may be a keyboard, mouse, touch screen, track pad, track ball, isopoint, and/or a voice recognition system. In one embodiment, wherein the device 100 is designed to be operated or interacted with only via remote devices, the device 100 may not include input devices 114. In other embodiments, input devices 114 include one or more storage devices, such as one or more flash drives, hard disk drives, solid state drives, cloud storage, or other storage devices or solutions, which provide data input to the host device 100.

One or more storage devices 118 may also be connected to the main unit 104 via the interface circuit 112. For example, a hard drive, CD drive, DVD drive, flash drive, and/or other storage devices may be connected to the main unit 104. The storage devices 118 may store any type of data used by the device 100, including data regarding preferred nutrient ranges, data regarding nutrient contents of various food items, data regarding users of the system, data regarding previously-generated dietary intake scores, data regarding previously-generated menus, recipes or meals, individual user preferences for menus, recipes or meals, frequency of preferences for menus, recipes or meals, data regarding ideal energy intake, data regarding past energy consumption, and any other appropriate data needed to implement the disclosed system, as indicated by block 150.

In several embodiments, the Recommendation System indicated by block 150 may store different database modules which include: a food database module; a menu database module (for example with: breakfast, lunch, dinner and snacks); a recipe database module; a dietary constraints module (for example with gluten-free, lactose-free, Mediterranean diet, vegetarian diet, vegan diet recommendations based on the dietary constraints); a nutrient scoring module (for example for determining the macronutrient or micronutrient score per menu, per recipe or per day); and/or optimization module.

Alternatively or in addition, storage devices 118 may be implemented as cloud-based storage, such that access to the storage 118 occurs via an internet or other network connectivity circuit such as an Ethernet circuit 112.

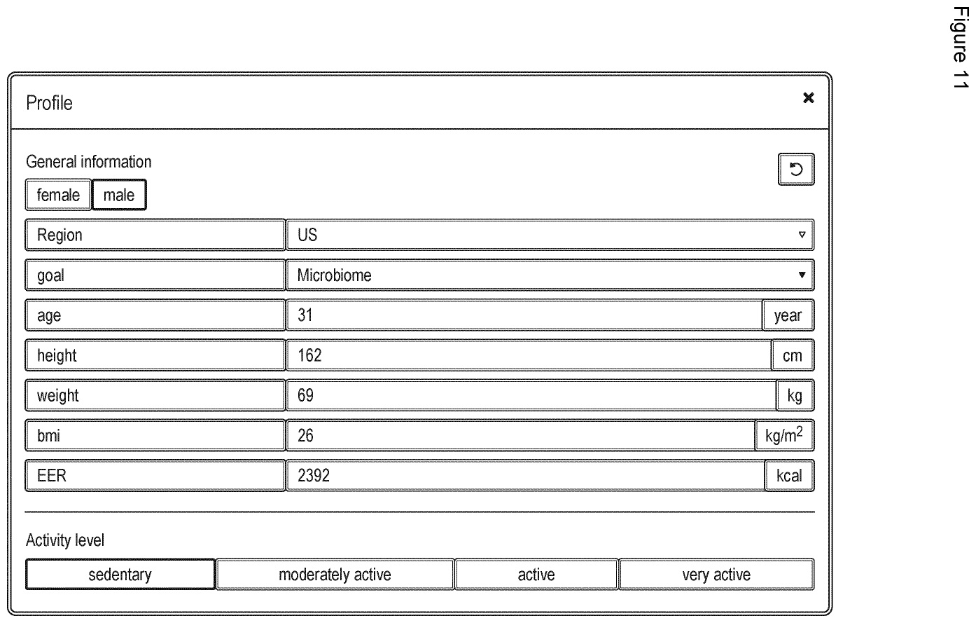

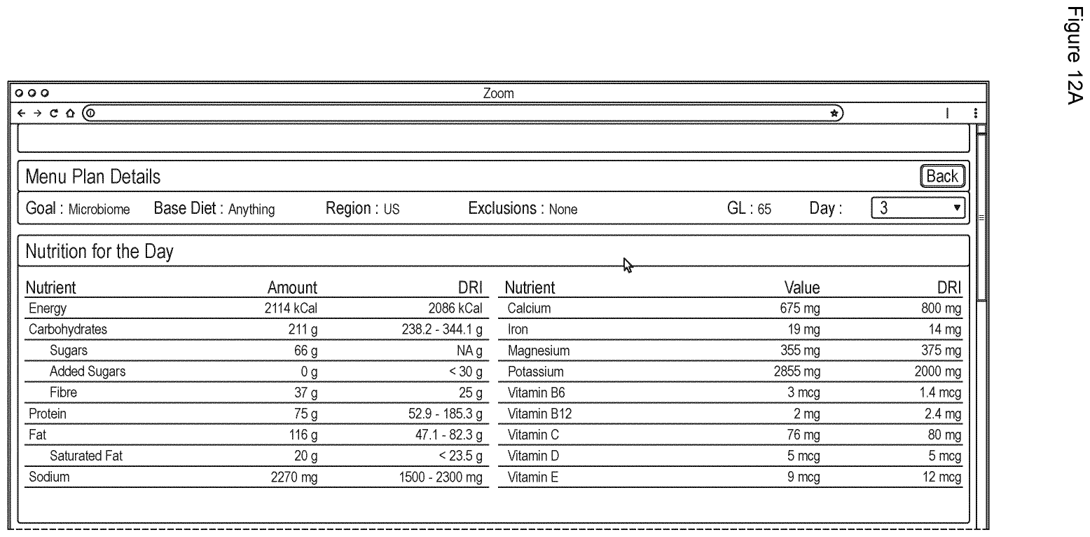

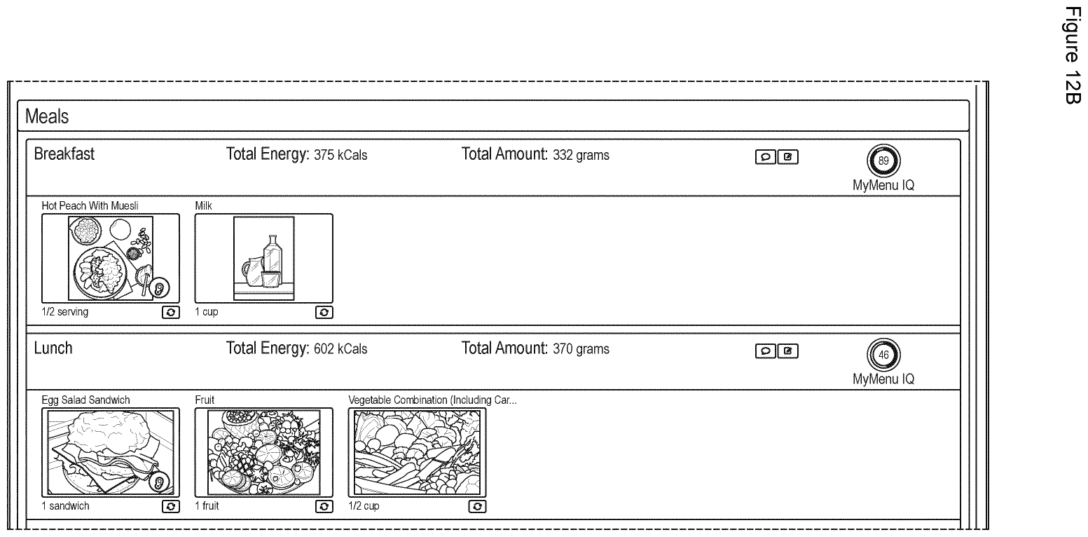

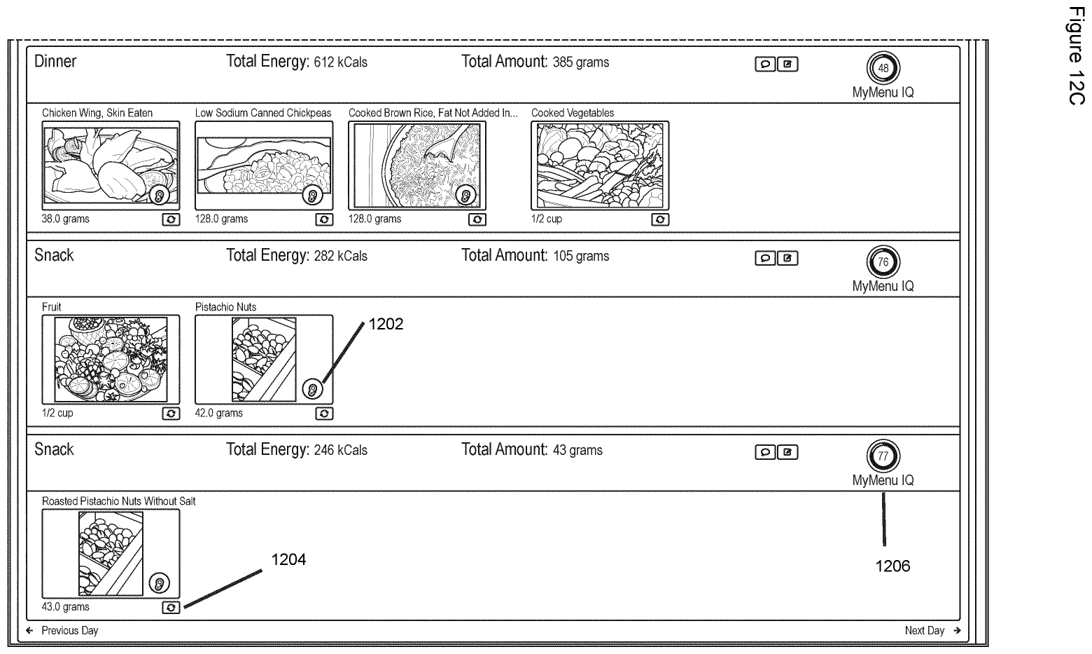

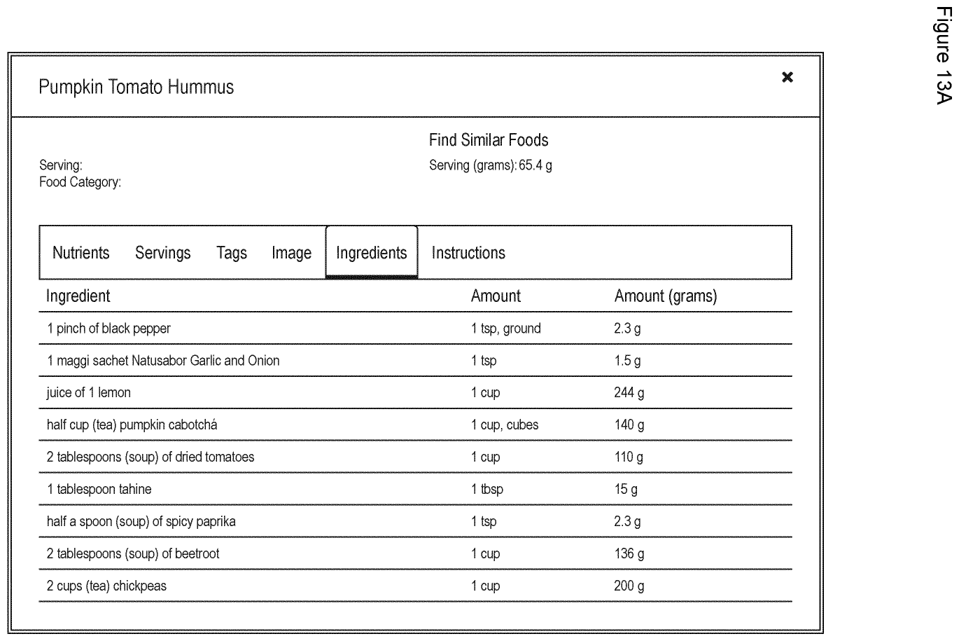

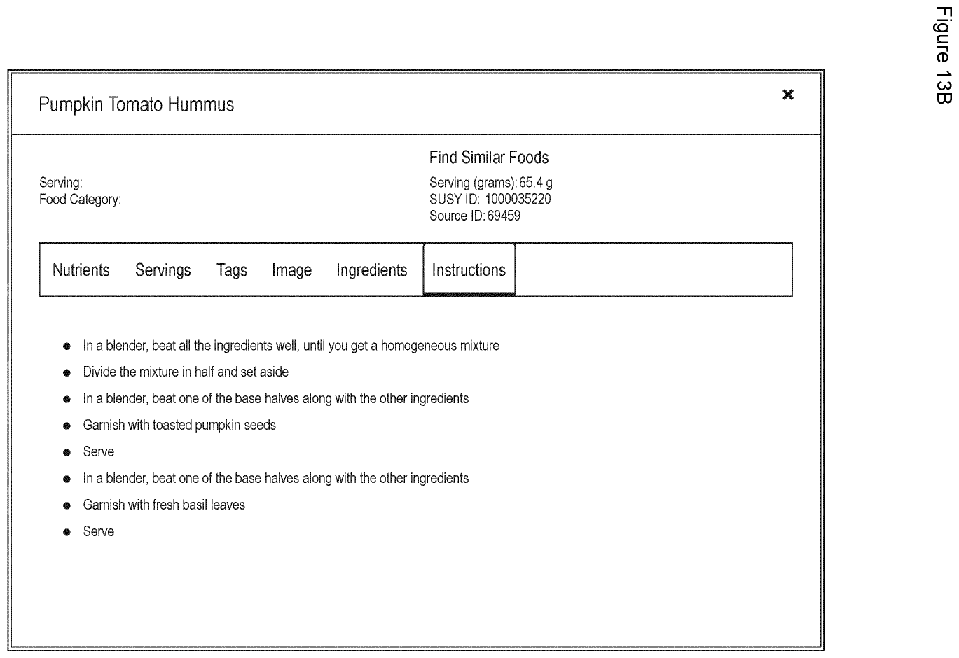

One or more displays 120, and/or printers, speakers, or other output devices 119 may also be connected to the main unit 104 via the interface circuit 112. The display 120 may be a liquid crystal display (LCD), a suitable projector, or any other suitable type of display. The display 120 generates visual representations of various data and functions of the host device 100 during operation of the host device 100. For example, the display 120 may be used to display information about the database of preferred nutrient ranges, a database of nutrient contents of various food items, a database of users of the system, a database of previously-generated menus, recipes or meals, and/or databases to enable an administrator at the device 100 to interact with the other databases described above. For example, as shown in FIG. 11 there is individual user information. In FIGS. 12A, 12B and 12C, there is a typical menu plan for one day. In FIG. 13, there is a typical microbiome-healthy recipe and instructions how to make it.

In the illustrated embodiment, the users of the computerized recommendation system interact with the device 100 using a suitable client device, such as client device 102. The client device 102 in various embodiments is any device that can access content provided or served by the host device 100. For example, the client device 102 may be any device that can run a suitable web browser to access a web-based interface to the host device 100. Alternatively or in addition, one or more applications or portions of applications that provide some of the functionality described herein may operate on the client device 102, in which case the client device 102 is required to interface with the host device 100 merely to access data stored in the host device 100, such as data regarding healthy nutrient ranges or nutrient content of various food items.

In one embodiment, this connection of devices (i.e., the device 100 and the client device 102) is facilitated by a network connection over the Internet and/or other networks, illustrated in FIG. 1 by cloud 116. The network connection may be any suitable network connection, such as an Ethernet connection, a digital subscriber line (DSL), a WiFi connection, a cellular data network connection, a telephone line-based connection, a connection over coaxial cable, or another suitable network connection.

In one embodiment, host device 100 is a device that provides cloud-based services, such as cloud-based authentication and access control, storage, streaming, and feedback provision. In this embodiment, the specific hardware details of host device 100 are not important to the implementer of the disclosed system-instead, in such an embodiment, the implementer of the disclosed system utilizes one or more Application Programmer Interfaces (APIs) to interact with host device 100 in a convenient way, such as to enter information about the user's demographics to help determine healthy nutritional ranges, to enter information about consumed foods, and other interactions described in more detail below.

Access to device 100 and/or client device 102 may be controlled by appropriate security software or security measures. An individual user's access can be defined by the device 100 and limited to certain data and/or actions, such as selecting various menus or recipes or viewing calculated scores, according to the individual's identity. Other users of either host device 100 or client device 102 may be allowed to alter other data, such as weighting, sensitivity, or healthy range values, depending on those users' identities. Accordingly, users of the system may be required to register with the device 100 before accessing the content provided by the disclosed system.

In a preferred embodiment, each client device 102 has a similar structural or architectural makeup to that described above with respect to the device 100. That is, each client device 102 in one embodiment includes a display device, at least one input device, at least one memory device, at least one storage device, at least one processor, and at least one network interface device. It should be appreciated that by including such components, which are common to well-known desktop, laptop, or mobile computer systems (including smart phones, tablet computers, and the like), client device 102 facilitates interaction among and between each other by users of the respective systems.

In various embodiments, devices 100 and/or 102 as illustrated in FIG. 1 may in fact be implemented as a plurality of different devices. For example, the device 100 may in actuality be implemented as a plurality of server devices operating together to implement the media content access system described herein. In various embodiments, one or more additional devices, not shown in FIG. 1, interact with the device 100 to enable or facilitate access to the system disclosed herein. For example, in one embodiment the host device 100 communicates via network 116 with one or more public, private, or proprietary repositories of information, such as public, private, or proprietary repositories of nutritional information, nutrient content information, menu planners, recipe databases, healthy range information, energy information, environmental impact information, or the like.

In one embodiment, the disclosed system does not include a client device 102. In this embodiment, the functionality described herein is provided on host device 100, and the user of the system interacts directly with host device 100 using input devices 114, display device 120, and output devices 119. In this embodiment, the host device 100 provides some or all of the functionality described herein as being user-facing functionality.

In various embodiments, the system disclosed herein is arranged as a plurality of modules, wherein each module performs a particular function or set of functions. The modules in these embodiments could be software modules executed by a general purpose processor, software modules executed by a special purpose processor, firmware modules executing on an appropriate, special-purpose hardware device, or hardware modules (such as application specific integrated circuits (“ASICs”)) that perform the functions recited herein entirely with circuitry. In embodiments where specialized hardware is used to perform some or all of the functionality described herein, the disclosed system may use one or more registers or other data input pins to control settings or adjust the functionality of such specialized hardware.

A user goal to eat a microbiome healthy diet may be examined over time to detect potential problematic menus or meals within the diet. The system can be used to then identify the recommended shifts needed in the food items, menus or recipes in order to get closer to the recommended amounts. In some embodiments, the system and methods disclosed herein can be used by nutritionists, health-care professionals, and individual users (e.g., users of wearable devices such as smart watches or fitness trackers).

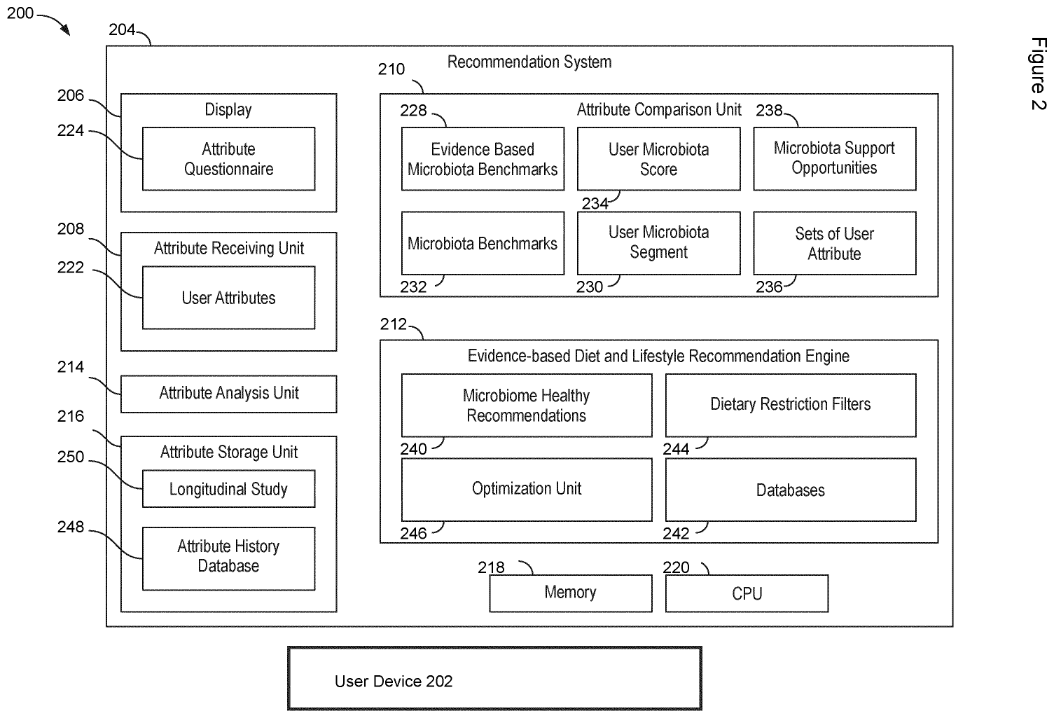

FIG. 2 illustrates a microbiome recommendation system according to an embodiment of the present disclosure. The system 200 includes a user device 202 and a recommendation system 204. In another embodiment of the present disclosure, the recommendation system 204 can be one example of the embodiment of the recommendation system 150 of FIG. 1. The user device 202 may be implemented as a computing device, such as a computer, smartphone, tablet, smartwatch, or other wearable through which an associated user can communicate with the recommendation system 204. The user device 202 may also be implemented as, e.g., a voice assistant configured to receive voice requests from a user and to process the requests either locally on a computer device proximate to the user or on a remote computing device (e.g., at a remote computing server).

The recommendation system 204 includes one or more of a display 206, an attribute receiving unit 208, an attribute comparison unit 210, an evidence-based diet and lifestyle recommendation engine 212, an attribute analysis unit 214, an attribute storing unit 216, a memory 218, and a CPU 220. Note, that in some embodiments, a display 206 may additionally or alternatively be located within the user device 202. In an example, the recommendation system 204 may be configured to receive a request for a plurality of microbiome-healthy recommendations 240. For example, a user may install an application on the user device 202 that requires the user to sign up for a recommendation service. By signing up for the service, the user device 202 may send a request for the microbiome-healthy recommendations 240. In a different example, the user may use the user device 202 to access a web portal using user-specific credentials. Through this web portal, the user may cause the user device 202 to request microbiome healthy recommendations from the recommendation system 204.

In another example, the recommendation system 204 may be configured to request and receive a plurality of user attributes 222. For example, the display 206 may be configured to present an attribute questionnaire 224 to the user. The attribute receiving unit 208 may be configured to receive the user attributes 222. In one example, the attribute receiving unit 208 may receive a plurality of answers 226 based on the attribute questionnaire 224, and based on the plurality of answers, determine the plurality of user attributes 222. For example, the attribute receiving unit 208 may receive answers to the attribute questionnaire 224 suggesting that the diet of the user is equivalent to the recommended dietary allowance (“RDA”) and then determine the user attributes 222 to be equivalent to the RDA, of Vitamin K per day. In another example, the user device attribute receiving unit 208 may directly receive the user attributes 222 from the user device 102.

In another example, the attribute receiving unit 208 may be configured to receive the test results of a home-test kit, the results of a standardized health test administered by a medical professional, the results of a self-assessment tool used by the user, or the results of any external or third party test. Based on the results from any of these tests or tools, the attribute receiving unit 208 may be configured to determine the user attributes 222. For example, the microbiome health status of the user may be determined before the intervention of the microbiome-healthy recommendations by measuring alpha-diversity of microbiota species, butyrate producing bacteria or short chain fatty acid production in the gut. The same measurements may be determined at a time period after the microbiome-healthy interventions to determine whether there has been an improvement or maintenance of the microbiome health status of the user.

The recommendation system 204 may be further configured to compare the plurality of user attributes 222 to a corresponding plurality of evidence-based microbiome-healthy benchmarks 228.

Furthermore, the attribute comparison unit 210 may be further configured to determine a microbiota benchmark set 232 based on the user's microbiota segment 230. For example, if the attribute comparison unit 210 determines that a user falls into the obese BMI segment 230, based on the plurality of user attributes 222, the attribute comparison unit 210 may select a microbiota benchmark set 232 that has been created and defined according to the specific needs for a healthy microbiome.

The comparison unit 210 may be further configured to select, from this determined microbiome benchmark set 232, the evidence-based microbiota benchmarks 128 and compare the now selected evidence-based microbiota benchmarks 228 to each of the corresponding user attributes 222. For example, when the microbiota benchmark set 232 has been determined, in response to the determination, the attribute comparison unit 210 may compare a user attribute 222 that represents the user's vitamin K intake to an evidence based microbiota benchmark 228 that represents a benchmark vitamin K intake, determining whether the user is below, at, or above the benchmark vitamin K intake. Though this example is based on a concrete, numerical comparison, another example of a benchmark comparison may be qualitative and different depending on a person. For example, a user attribute 222 may indicate that the user is currently experiencing higher than normal levels of stress. An example benchmark related to a user stress level may indicate that an average or low level of stress is desired and thus, the user attribute 222 indicating a higher level of stress is determined to be below that of the benchmark. As different users experience differing levels of stress, even under the same circumstances, such a comparison requires a customized approach.

In addition, during the comparison from the prior example, the attribute comparison unit 210 may be configured to determine a user microbiota score 234 based on the comparison between the evidence-based microbiota benchmarks 228 and the user attributes 222. For example, the attribute comparison unit 210 may determine a user microbiota score of 95/100 if the user attributes 222 very nearly meet all or most of the corresponding evidence-based microbiota benchmarks 228. In another example, a score may be represented through lettering grades, symbols, or any other system of ranking, for example, “high”, “average”, “low” that allows a user to interpret how well their current attributes rate amongst benchmarks. This user microbiota score 234 may be presented through the display 206.

The recommendation system 204 may be further configured to determine a plurality of microbiota support opportunities 238 based on the plurality of user attributes 222 and the comparison to the corresponding plurality of evidence-based microbiota benchmarks 228. In one example, the attribute comparison unit 210 may determine microbiota support opportunities 238 for every user attribute 222 that does not meet the corresponding evidence-based microbiota benchmark. In this example, a corresponding evidence-based microbiota benchmark 228 may require a user have an intake of 2 ug/day of folate, whereas the user attribute may indicate the user is only receiving 1 ug/day of folate. Therefore, the attribute comparison unit 210 may determine an increase in folate intake to be a microbiota support opportunity 238.

In another example, the attribute comparison unit 210 may be configured to identify a first set of user attributes 236 comprised of each of the plurality of user attributes 222 that are below the corresponding one of the plurality of evidence-based microbiota benchmarks 228 as well as identify a second set of user attributes 236 comprised of each of the plurality of user attributes 222 that are greater than or equal to the corresponding evidence-based microbiota benchmarks 228. While the first set of user attributes 236 is determined similarly to the above given example, the second set of user attributes 236 differs in that, although the associated user does not appear to have a deficiency, there may be opportunities to support microbiome health by recommending the user maintain current practices or opportunities to further improve upon them. Accordingly, the recommendation system 204 may determine opportunities to support microbiome health based on which attributes 222 populate either sets 236.

The recommendation system 204 may be further configured to identify a plurality of microbiome-healthy recommendations 240 based on the plurality of microbiota support opportunities 238. For example, the evidence-based diet and lifestyle recommendation engine 212 may be configured to be cloud-based. The recommendation engine 212 may comprise one or more of a plurality of databases 242, a plurality of dietary restriction filters 244, and an optimization unit 246. Based on the plurality of opportunities 238, the recommendation engine 212 may identify the plurality of microbiome-healthy recommendations 240 according to the one or more of plurality of databases 242, the dietary restriction filters 244, and the optimization unit 246.

In another example, the recommendation system 204 may be configured to provide continuous recommendations, based on prior user attributes. For example, the recommendation system 204 may comprise, in addition to the previously discussed elements, an attribute storing unit 216 and an attribute analysis unit 214. The attribute storing unit 216 may be configured to, responsive to the attribute receiving unit 108 receiving the plurality of user attributes 222, add the received user attributes 222 to an attribute history database 248 as a new entry based on when the plurality of user attributes 222 were received. For example, if user attributes 222 are received by the attribute receiving unit 208 on a first day, the attribute storing unit 216 will add the received user attributes 222 to a cumulative attribute history database 248 noting the date of entry, in this case the first day. Later, if user attributes 222 are received by the attribute receiving unit 208 on a second day, e.g. the next day, the attribute storing unit 216 will also add these new attributes to the attribute history database 248, noting that they were received on the second day, while also preserving the earlier attributes from the first day.

This attribute analysis unit 214 may be configured to analyze the plurality of user attributes 222 stored within the attribute history database 248, wherein analyzing the stored plurality of user attributes 222 comprises performing a longitudinal study 250. Continuing the earlier example, the attribute analysis unit 214 may perform a longitudinal study of the user attributes 222 from each of the first day, the second day, and every other collection of user attributes 222 found within the attribute history database 248. The evidence based diet and lifestyle recommendation engine 212 may be further configured to generate a plurality of microbiome-healthy recommendations 240 based on at least the stored user attributes 222 found within the attribute history database 248 and the analysis performed by the attribute analysis unit 214.

In an embodiment, the attribute analysis unit 214 is further configured to repeatedly analyze the plurality of user attributes 222 stored within the attribute history database 248 responsive to the attribute storing unit 216 adding a new entry to the attribute history database 248, essentially re-analyzing all of the data within the attribute history database 248 immediately after new user attributes 222 are received. Similarly, the evidence based diet and lifestyle recommendation engine 212 may be further configured to repeatedly generate the plurality of microbiome-healthy recommendations 240 responsive to the attribute analysis unit 214 completing an analysis, thereby effectively generating new microbiome-health recommendations 240 that consider all past and present user attributes 222 each time a new set of user attributes 222 is received.

In various embodiments, the user-specific (or population-specific) inputs to the disclosed system are programmable and configurable, and include gender, age, weight, height, physical activity level, whether obese, and the like. For example, FIG. 11 shows typical individual user data.

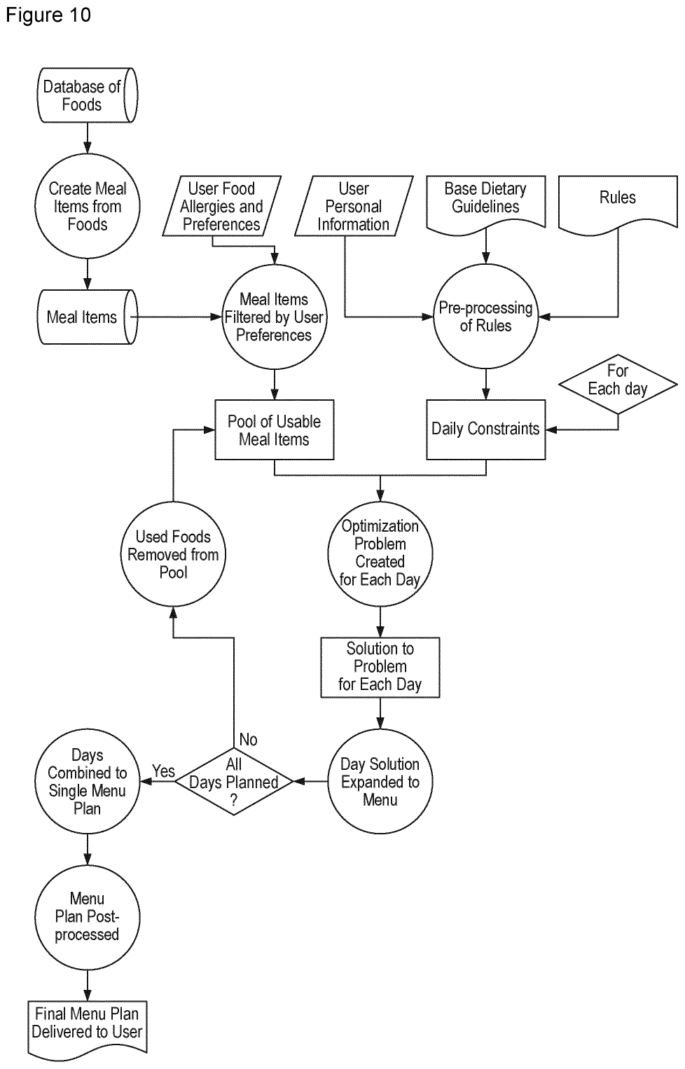

In an embodiment, the disclosed system includes or is connected to a database containing foods items, menus or recipes and respective nutrient content. In this embodiment, the disclosed system includes a fuzzy search feature that enables a user to enter a consumed (or to-be consumed) food, and thereafter searches the database to find a closest item to the user-provided item. The disclosed system, in this embodiment, uses stored nutritional information about the matched food item to determine whether it is a microbiome-healthy item. For example, FIG. 10 shows an example of the workflow for a microbiome-healthy menu plan.

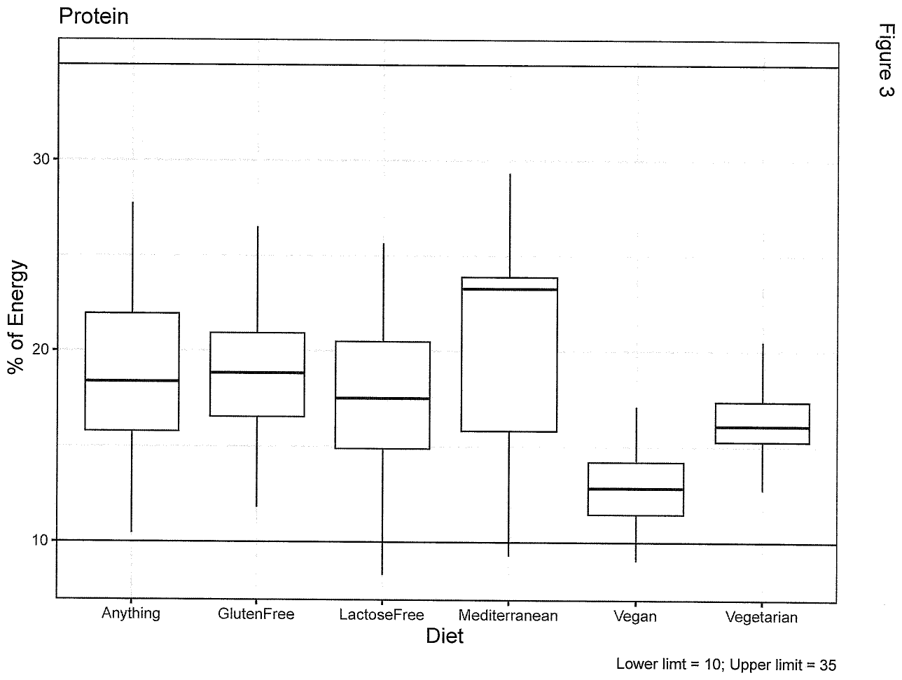

In various embodiments, the disclosed system further includes an interface (e.g., a graphical user interface) to display the amount of each nutrient available in each food composing the diet, and displays the amount of energy available to be consumed. For example in FIGS. 3 to 9, the nutrients have been balanced for different diet preferences such as: no restrictions, gluten-free, lactose-free, Mediterranean diet, vegan diet or vegetarian diet. In some embodiments, this interface enables users to modify the amount of various foods or energy to be consumed. In other embodiments, the system is configured to determine amounts of food or energy consumed using non-user-input data, such as by scanning one or more bar codes, QR codes, or RFID tags, image recognition systems, or by tracking items ordered from a menu or purchased at a grocery store.

Various embodiments of the disclosed system display a dashboard or other appropriate user interface to a user that is customized based on the user's needs. In embodiments of the system disclosed herein, a graphical user interface is provided which advantageously enables, for the first time, users to input data about food consumed in a given period of time and to see an indication of a score, based appropriately on energy consumption, that reflects overall nutritional content of the consume diet.

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1

### Nutritional Foundation And Nutritional Constraints of the Menu Planner Algorithm

The nutritional foundation of the meal planner was built upon the dietary reference intakes set by the World Health Organization (WHO) and the U.S. Institute of Medicine (IoM), as well as “Dietary Guidelines for Americans” published by the US Department of Agriculture (USDA). Various levels of personalization were applied based on phenotype:

(i) micronutrient Recommended Daily Allowances (RDAs) depend on gender and age group, as well as specific medical conditions (e.g., pregnancy or lactation);

(ii) macronutrient RDAs were expressed as a fraction of daily energy requirement;

(iii) the estimated energy requirement was computed based on biometric information (gender, age, weight, and height), as well as estimated average activity level (sedentary, moderately active, active, very active);

The energy requirements were based on the Institute of Medicine (IOM) equation (https://en.wikipedia.org/wiki/Institute_of_Medicine_Equation, 2002) in the context of Dietary Guidelines for Americans (DGA) (https://health.gov/dietaryguidelines/2015/guidelines/table-of-contents/, 2015). A correction factor is applied for overweight and obese individuals (body mass index ≥25) based on AHA/ACC/TOS, «Guideline for the Management of Overweight and Obesity in Adults» Circulation, 2013 and Frankenfield (2013) Clin. Nutr., vol. 32, no. 16, p. 976-982, 2013.

After stablishing the nutritional foundations of the algorithm, the menu planner followed the Healthy U.S. Style Patterns, which were based on the types and proportions of foods Americans typically consume, but in nutrient-dense forms and appropriate amounts. With the information captured in Healthy US style eating pattern, desired nutrition goals were achieved by selecting foods from multiple groups, such as vegetables, fruits, grains, dairy, protein foods and oils. These guidelines were designed to meet nutrient needs while not exceeding calorie requirements and while staying within limits for overconsumed dietary components, the outputs of the menu plan algorithm closely followed the dietary guidelines and recommendations in the US.

2000 kcal was a reference diet to implement the microbiome rules according to the guidelines for the corresponding food group. Table 1 below shows the recommended food groups and quantity of consumption on weekly and daily basis, for a 2000 kcal daily diet.

TABLE 1
Recommendation for a 2000 kcal/day diet

| Food group | Recommended for 2,000 kcal/day | |
|---|---|---|
| Vegetables | 2½ | cup equivalents |
| Dark-green vegetables (c-eq/wk) | 1½ | |
| Red and orange vegetables (c-eq/wk) | 5½ | |
| Legumes (beans and peas) (c-eq/wk) | 1½ | |
| Starchy vegetables (c-eq/wk) | 5 | |
| Other vegetables (c-eq/wk) | 4 | |
| Fruits | 2 | cup equivalents |
| Grains | 6 | oz equivalents |
| Wholegrains (oz-eq/day) | 3 | |
| Refined grains (oz-eq/day) | 3 | |
| Dairy | 3 | cup equivalents |
| Protein Foods | 5½ | oz equivalents |
| Seafood (oz-eq/wk) | 8 | |
| Meats, poultry, eggs (oz-eq/wk) | 26 | |
| Nuts seeds, soy products (oz-eq/wk) | 5 | |
| Oils | 27 | g |

Nutrition requirements of the individual was highly dependent upon the age, sex and physical activity and other factors. With respect to individual nutrition needs, the menu plan followed the dietary guidelines for calories, macronutrient requirements and dietary fiber for each age and sex group.

TABLE 2
Daily Nutritional Goals for Age-Sex Groups Based on Dietary
Reference Intakes & Dietary Guidelines Recommendations
Reference: https://health.gov/sites/default/files/2019-09/2015-2020 Dietary Guidelines.pdf

| | | Child | Female | Male | Female | Male | Female | Male |
|---|---|---|---|---|---|---|---|---|
| | Source | 1-3 | 4-8 | 4-8 | 9-13 | 9-13 | 14-18 | 14-18 |
| Calorie | | 1,000 | 1,200 | 1,400, | 1,600 | 1,800 | 1,800 | 2,200, |
| Level(s) | | | | 1,600 | | | | 2,800, |
| Assessed | | | | | | | | 3,200 |
| Macro- | | | | | | | | |
| nutrients | | | | | | | | |
| Protein, g | RDA | 13 | 19 | 19 | 34 | 34 | 46 | 52 |
| Protein, | AMDR | 5-20 | 10-30 | 10-30 | 10-30 | 10-30 | 10-30 | 10-30 |
| % kcal | | | | | | | | |
| Carbo- | RDA | 130 | 130 | 130 | 130 | 130 | 130 | 130 |
| hydrate, g | | | | | | | | |
| Carbo- | AMDR | 45-65 | 45-65 | 45-65 | 45-65 | 45-65 | 45-65 | 45-65 |
| hydrate, | | | | | | | | |
| % kcal | | | | | | | | |
| Dietary | 14 g/ | 14 | 16.8 | 19.6 | 22.4 | 25.2 | 25.2 | 30.8 |
| Fiber, g | 1,000 | | | | | | | |
| | kcal | | | | | | | |
| Added | DGA | <10% | <10% | <10% | <10% | <10% | <10% | <10% |
| Sugars, | | | | | | | | |
| % kcal | | | | | | | | |
| Total Fat, | AMDR | 30-40 | 25-35 | 25-35 | 25-35 | 25-35 | 25-35 | 25-35 |
| % kcal | | | | | | | | |

| | | Female | Male | Female | Male | Female | Male |
|---|---|---|---|---|---|---|---|
| | Source | 19-30 | 19-30 | 31-50 | 31-50 | 51+ | 51+ |
| Calorie | | 2,000 | 2,400, | 1,800 | 2,200 | 1,600 | 2,000 |
| Level(s) | | | 2,600, | | | | |
| Assessed | | | 3,000 | | | | |
| Macro- | | | | | | | |
| nutrients | | | | | | | |
| Protein, g | RDA | 46 | 56 | 46 | 56 | 46 | 56 |
| Protein, | AMDR | 10-35 | 10-35 | 10-35 | 10-35 | 10-35 | 10-35 |
| % kcal | | | | | | | |
| Carbo- | RDA | 130 | 130 | 130 | 130 | 130 | 130 |
| hydrate, g | | | | | | | |
| Carbo- | AMDR | 45-65 | 45-65 | 45-65 | 45-65 | 45-65 | 45-65 |
| hydrate, | | | | | | | |
| % kcal | | | | | | | |
| Dietary | 14 g/ | 28 | 33.6 | 25.2 | 30.8 | 22.4 | 28 |
| Fiber, g | 1,000 | | | | | | |
| | kcal | | | | | | |
| Added | DGA | <10% | <10% | <10% | <10% | <10% | <10% |
| Sugars, | | | | | | | |
| % kcal | | | | | | | |
| Total Fat, | AMDR | 25-35 | 25-35 | 25-35 | 25-35 | 25-35 | 25-35 |
| % kcal | | | | | | | |

### Example 2

### Microbiome Healthy Food Rules and Implementation in a Menu Plan

More than 1400 scientific articles were analyzed to find food ingredients and food compounds rules that could be directly applied to the menu planner engine. A database was created with food compound rules and food ingredient rules. However, it was not possible to directly implement the food ingredient rules into the menu planner engine without modification and only the food compound rules could be adapted and implemented in the engine.

The Table 3 below lists the final rules implemented into the menu planner. In column 2, “Rules implemented”, were the actual rules which were implemented into the menu planner and with which frequency. In column 3, “Rule quantities in literature” were the rules as found in the literature. In column 4, “Adapted rules” were the rules adapted to fit a healthy microbiome menu plan, feasible to be used by the consumer.

TABLE 3
Food or Nutrient Rules Implemented into Menu Planner

| | | Rule | | | | | | Source | | |
|---|---|---|---|---|---|---|---|---|---|---|
| | | quantities | | | | | | on | Source | |
| Food/ | Rules | in | | Adapted | | Normal | | publi- | Prep. | |
| nutrient | implemented | literature | | rules | | portion size | | cation | method | Ref. |
| Nutrient | fibre from | 18.7 g fiber | | 490 g and/or | | 140 g cooked; | | raw | cooked | 1 |
| | barley or fiber | (60 g) | | 240 g (mostly | | 45 g dry | | flakes | | |
| | from brown | and/or 4.4 g | | barley in | | | | | | |
| | rice once | fiber (60 g) | | combination | | | | | | |
| | every 3 days, | | | with rice) | | | | | | |
| | starting on | | | | | | | | | |
| | day 1 | | | | | | | | | |
| Food | chickpeas | 200 | g | 200 | g | 130 g canned; | | canned; | cooked | 2 |
| | once every 6 | | | | | 35 g dry | | cooked | | |
| | days, starting | | | | | | | | | |
| | on day 2 | | | | | | | | | |
| Food | pistachios | 86 | g | 86 | g | 30 | g | nuts | raw | 3 |
| | every 12 days | | | | | | | | | |
| | (OPTIONAL), | | | | | | | | | |
| | starting on | | | | | | | | | |
| | day 3 | | | | | | | | | |
| Nutrient | EPA and | 2 g EPA | | 200-300 g | | 85-110 | g fish | supple- | cooked | 4 |
| | DHA, once | and 2 g | | (depending on | | | | ments | | |
| | every 6 days, | DHA | | fish type) | | | | | | |
| | starting on | | | | | | | | | |
| | day 5 | | | | | | | | | |
| Food | walnuts once | 42 | g | 42 | g | 30 | g | nuts | raw | 5 |
| | every 6 days | | | | | | | | | |
| | (OPTIONAL), | | | | | | | | | |
| | starting on | | | | | | | | | |
| | day 6 | | | | | | | | | |
| Food | almonds once | 85 | g | 85 | g | 30 | g | nuts | raw | 6 |
| | every 12 days | | | | | | | | | |
| | (OPTIONAL), | | | | | | | | | |
| | starting on | | | | | | | | | |
| | day 9 | | | | | | | | | |
| Nutrient | fibre from rye | 16 | g fiber | 300 g or 150 g | | 50 | g | bread; | cooked | 7 |
| | once every 24 | | | (depending on | | | | cooked; | | |
| | days, starting | | | type of bread) | | | | 85% | | |
| | on day 12 | | | | | | | whole | | |
| | | | | | | | | rye | | |
| | | | | | | | | kernels | | |
| | | | | | | | | and 15% | | |
| | | | | | | | | white | | |
| | | | | | | | | wheat | | |
| | | | | | | | | flour | | |
| Food | of miso/natto, | 50 | g | 300 | mL | 15 g in 300 mL | | soup; | cooked | 8 |
| | once every 12 | | | | | | | cooked | | |
| | days, starting | | | | | | | | | |
| | on day 12 | | | | | | | | | |
| | (OPTIONAL) | | | | | | | | | |
| Food | flaxseeds | 0.3 | g/kg | 20 | g | 30 | g | seeds | raw | 9 |

| | once every 24 | | | | | | | | |
|---|---|---|---|---|---|---|---|---|---|
| | days, starting | | | | | | | | |
| | on day 24 | | | | | | | | |
| Food | wholegrain | 35 | g | 98 | g | 98 g = 3.5 oz | diet; | cooked | 10 |

| | |
|---|---|
| every day | cooked |

The menu planner was based on the US Healthy Dietary Patterns with the microbiome-healthy rules implemented on top so that the US Healthy Dietary Patterns were always applied. When designing the microbiome-healthy rules, several iterations were made to adjust the rules in order to keep the microbiome-healthy menus within US daily nutritional goals for age-sex group, based on dietary reference intakes and dietary guideline recommendations.

### REFERENCES

1. Martínez, I., Lattimer, J. M., Hubach, K. L., Case, J. A., Yang, J., Weber, C. G., & Haub, M. D. (2013). Gut microbiome composition is linked to whole grain-induced immunological improvements. The ISME journal, 7(2), 269-280.

2. Fernando, W., Hill, J., Zello, G., Tyler, R., Dahl, W., & Van Kessel, A. (2010). Diets supplemented with chickpea or its main oligosaccharide component raffinose modify faecal microbial composition in healthy adults. Beneficial microbes, 1(2), 197-207.

3. Ukhanova, M., Wang, X., Baer, D. J., Novotny, J. A., Fredborg, M., & Mai, V. (2014). Effects of almond and pistachio consumption on gut microbiota composition in a randomized cross-over human feeding study. British Journal of Nutrition, 111(12), 2146-2152.

4. Menni, C., Zierer, J., Pallister, T., Jackson, M. A., Long, T., Mohney, R. P., . . . & Valdes, A. M. (2017). Omega-3 fatty acids correlate with gut microbiome diversity and production of N-carbamylglutamate in middle aged and elderly women. Scientific reports, 7(1), 1-11.

5. Holscher, H. D., Guetterman, H. M., Swanson, K. S., An, R., Matthan, N. R., Lichtenstein, A. H., & Baer, D. J. (2018). Walnut consumption alters the gastrointestinal microbiota, microbially derived secondary bile acids, and health markers in healthy adults: a randomized controlled trial. The Journal of nutrition, 148(6), 861-867.

6. Ukhanova, M., Wang, X., Baer, D. J., Novotny, J. A., Fredborg, M., & Mai, V. (2014). Effects of almond and pistachio consumption on gut microbiota composition in a randomized cross-over human feeding study. British Journal of Nutrition, 111(12), 2146-2152.

7. Prykhodko, O., Sandberg, J., Burleigh, S., Björck, I., Nilsson, A., & Fåk Hållenius, F. (2018). Impact of Rye Kernel-Based Evening Meal on Microbiota Composition of Young Healthy Lean Volunteers With an Emphasis on Their Hormonal and Appetite Regulations, and Blood Levels of Brain-Derived Neurotrophic Factor. Frontiers in nutrition, 5, 45.

8. Fujisawa, T., Shinohara, K., Kishimoto, Y., & Terada, A. (2006). Effect of miso soup containing Natto on the composition and metabolic activity of the human faecal flora. Microbial ecology in health and disease, 18(2), 79-84.

9. Lagkouvardos, I., Kläring, K., Heinzmann, S. S., Platz, S., Scholz, B., Engel, K. H., & Clavel, T. (2015). Gut metabolites and bacterial community networks during a pilot intervention study with flaxseeds in healthy adult men. Molecular nutrition & food research, 59(8), 1614-1628.

10. Vanegas, S. M., Meydani, M., Barnett, J. B., Goldin, B., Kane, A., Rasmussen, H., & Koecher, K. (2017). Substituting whole grains for refined grains in a 6-wk randomized trial has a modest effect on gut microbiota and immune and inflammatory markers of healthy adults. The American journal of clinical nutrition, 105(3), 635-650.

### Example 3

### Macronutrient Nutritional Quality of the Microbiome-Healthy Menu Plan

The macronutrient nutritional quality of the microbiome-healthy menu plan was tested for a 28-day microbiome-healthy diet. In particular, the menu plan was tested for different dietary constraints: “anything” which was an omnivorous diet with no dietary restrictions, gluten-free diet, lactose-free diet, Mediterranean diet, vegan diet and vegetarian diet.

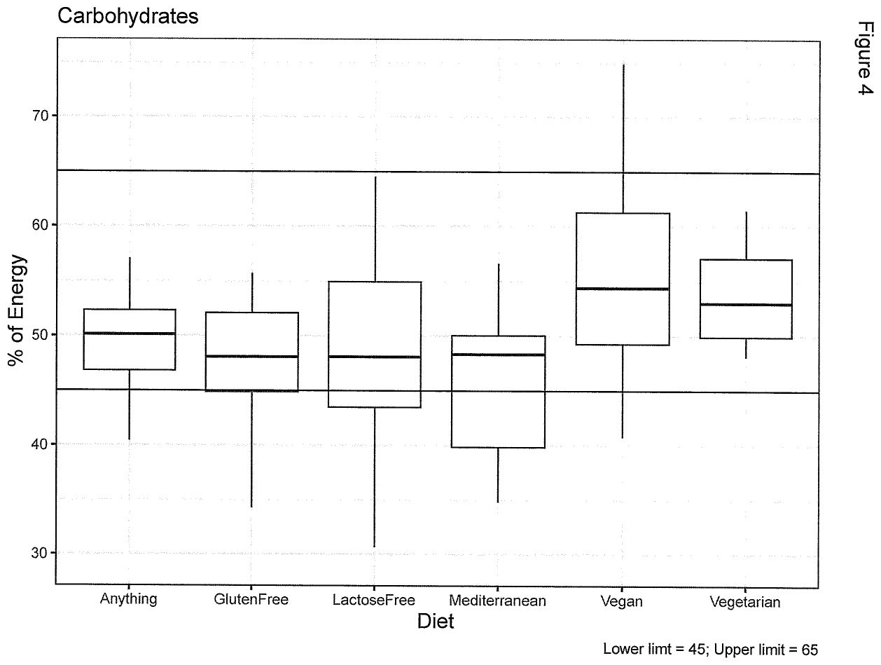

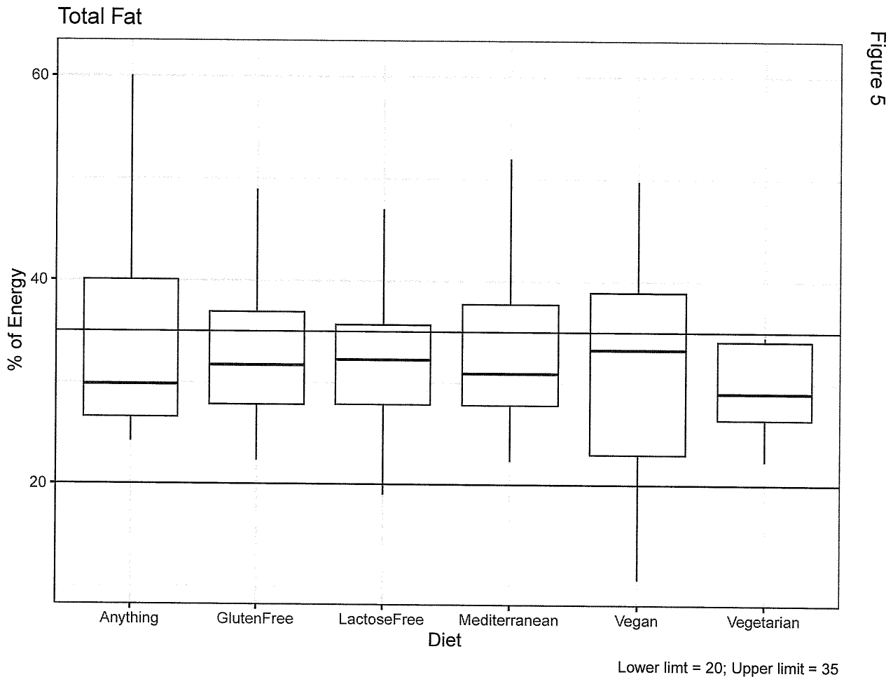

The results are shown individually for the macronutrients: protein in FIG. 3, carbohydrate in FIG. 4 and total fat in FIG. 5.

All microbiome-healthy diet menu plans fell within the ranges of daily macronutrients recommendations from US guidelines.

### Example 4

### Micronutrient Nutritional Quality of the Microbiome-Healthy Menu Plan

The micronutrient nutritional quality of the microbiome-healthy menu plan was tested for a 28-day microbiome-healthy diet. In particular, the menu plan was tested for different dietary constraints: “anything” which was an omnivorous diet with no dietary restrictions, gluten-free diet, lactose-free diet, Mediterranean diet, vegan diet and vegetarian diet.

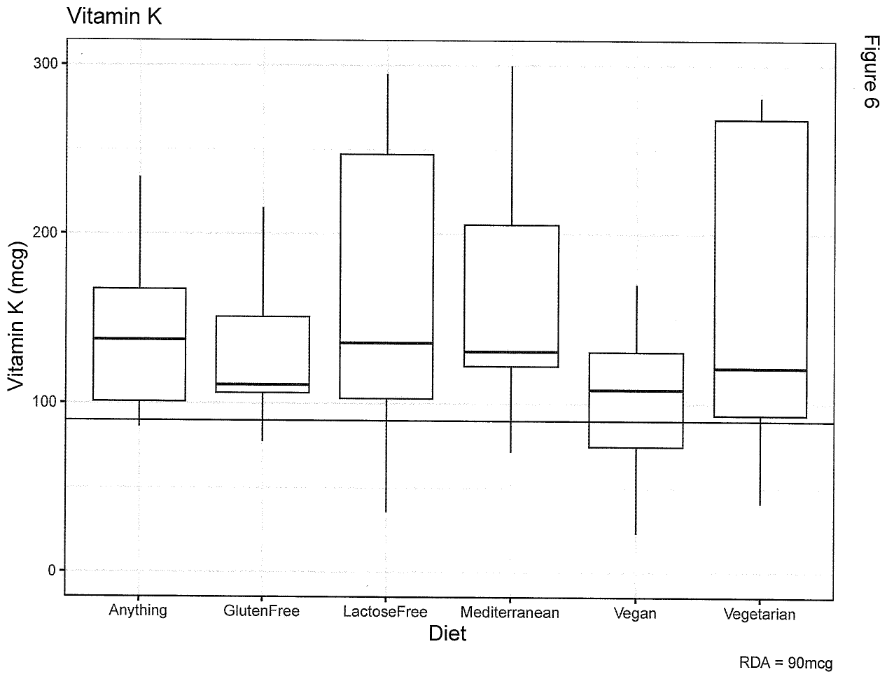

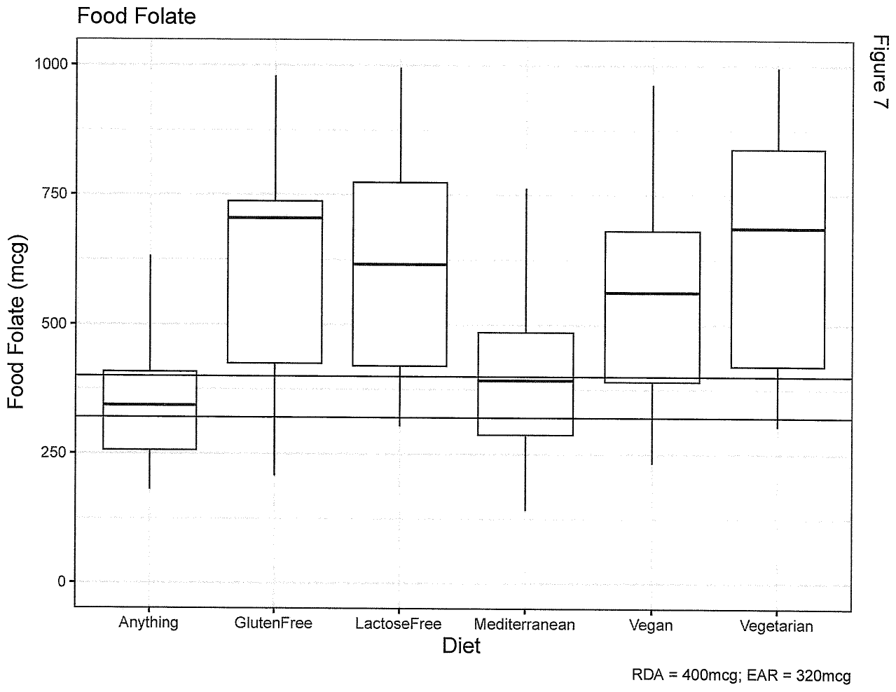

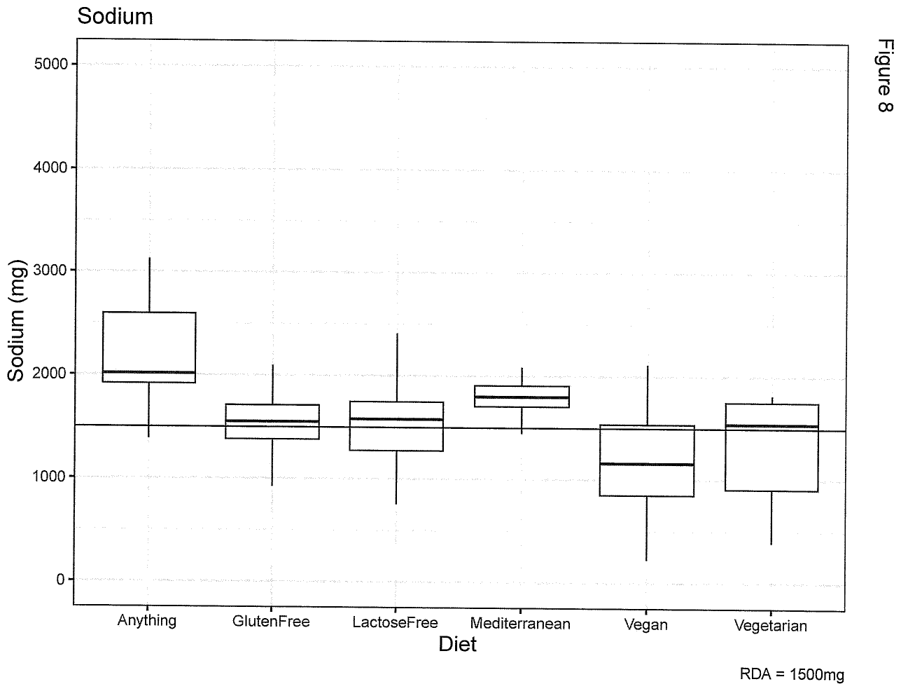

The results are shown for some key representative micronutrients: vitamin K in FIG. 6, food folate in FIG. 7 and sodium in FIG. 8.

The dietary constraints were included to the microbiome menu planners in order to respect the daily recommendations of micronutrients, in particular, vitamins and minerals. Estimated average requirements (EAR) were used to calculate the micronutrients necessities of the user, based on age and gender, as seen in the table of Dietary Reference Intakes (DRIs) below in Table 4. If EAR was not available for a specific nutrient, the Adequate Intake (Al) was used instead. The EAR was used instead of the RDA as the EAR is more broadly applicable to large populations.

The microbiome friendly menu planner fell within the ranges of daily micronutrients recommendations from US guidelines for all the given diets.

TABLE 4
Vitamin and Mineral recommendations from the US guidelines
Reference:
https://www.ncbi.nlm.nih.gov/books/NBK56068/table/summarytables.t1/?report=objectonly

| Age | | | | | | Ribo- | | | | |
|---|---|---|---|---|---|---|---|---|---|---|
| Group | Vit A | Vit C | Vit D | Vit E | Thiamin | flavin | Niacin | Vit B₆ | Folate | Vit. B₁₂ |
| (years) | (μg/d) | (mg/d) | (μg/d) | (mg/d) | (mg/d) | (mg/d) | (mg/d) | (mg/d) | (μg/d) | (μg/d) |
| Males | | | | | | | | | | |
| 19-70 | 75 | 10 | 12 | 1.0 | 1.1 | 12 | 1.1 | 320 | 2.0 | 700 |
| >70 | 75 | 10 | 12 | 1.0 | 1.1 | 12 | 1.4 | 320 | 2.0 | 700 |
| Females | | | | | | | | | | |
| 19-50 | 60 | 10 | 12 | 0.9 | 0.9 | 11 | 1.1 | 320 | 2.0 | 700 |
| 51->70 | 60 | 10 | 12 | 0.9 | 0.9 | 11 | 1.3 | 320 | 2.0 | 700 |

| Age | | | | | | | | |
|---|---|---|---|---|---|---|---|---|
| Group | Ca | Cu | I | Fe | Mg | P | Se | Zn |
| (years) | (mg/d) | (μg/d) | (μg/d) | (mg/d) | (mg/d) | (mg/d) | (μg/d) | (mg/d) |
| Males | | | | | | | | |
| 19-70 | 800 | 700 | 95 | 6 | 330 | 580 | 45 | 9.4 |
| >70 | 1000 | 700 | 95 | 6 | 350 | 580 | 45 | 9.4 |
| Females | | | | | | | | |
| 19-50 | 800 | 700 | 95 | 8.1 | 255 | 580 | 45 | 6.8 |
| 51->70 | 1000 | 700 | 95 | 5 | 265 | 580 | 45 | 6.8 |

### Example 5

### Total Fiber contribution to Nutritional Quality of the Microbiome-Healthy Menu Plan

Total fiber is considered to be one of the most important dietary ingredients for a healthy gut microbiota. For this reason, the targeted amount of fiber was set higher than the amounts of fiber in a usual average recommended daily intake (RDA=25 g/day).

The total fiber contribution to the nutritional quality of the microbiome-healthy menu plan was tested for a 28-day microbiome-healthy diet. In particular, the menu plan was tested for different dietary constraints: “anything” which was an omnivorous diet with no dietary restrictions, gluten-free diet, lactose-free diet, Mediterranean diet, vegan diet and vegetarian diet.

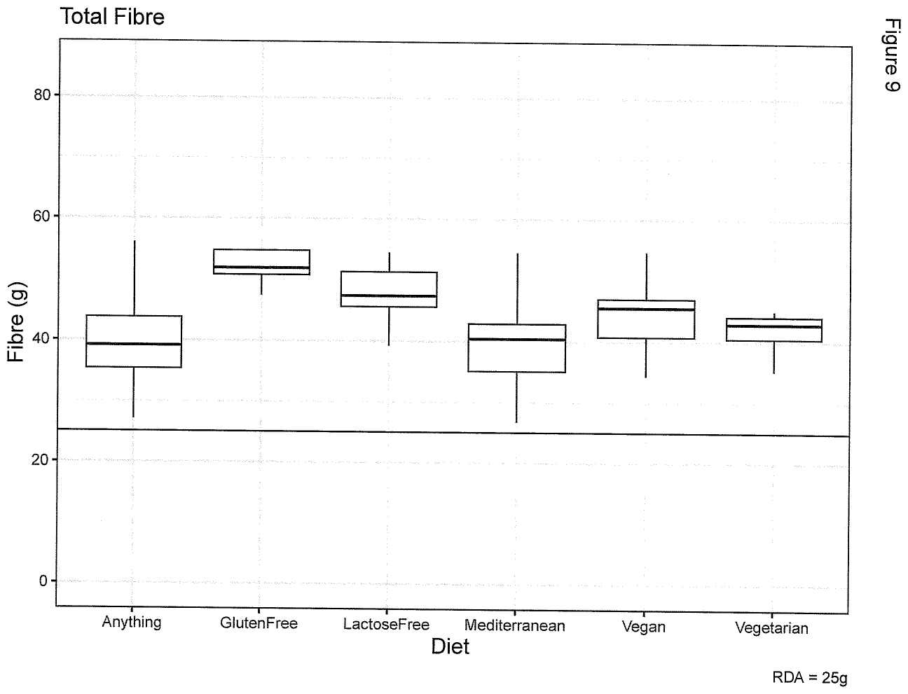

In FIG. 9, all the diets had a high content in fiber, in order to enhance microbiota health.

### Example 6

### Calculation of the Upper Limits for Different Micronutrients Present in the Menu Plan Based on the Microbiome-Healthy Rules

The upper tolerable intakes of the different micronutrients (vitamins and minerals) present in the microbiome healthy food ingredients were taken into account in order that the menu plan did not exceed the tolerable upper limits.

In Table 5 below, the different micronutrient quantities present in all the rules were represented against their tolerable upper limits. None of the rules surpassed the upper limits.

TABLE 5
Upper Tolerable Limits of Vitamins and Minerals

| Foods | vitD | vitB6 | vitB12 | vitB3 | vitC | vitE | vitB2 | vitK | vitA |
|---|---|---|---|---|---|---|---|---|---|
| Chickpeas, | 0 | 0.232 | 0 | 0.28 | 0.2 | 0.58 | 0.03 | 6.8 | 2 |
| mature | | | | | | | | | |
| seeds, | | | | | | | | | |
| canned, | | | | | | | | | |
| drained | | | | | | | | | |
| solids | | | | | | | | | |
| Barley, | 0 | 0.563 | 0 | 10.1087 | 0 | 0.049 | 0.303 | 3.92 | 0 |
| pearled, | | | | | | | | | |
| cooked | | | | | | | | | |
| Brown rice | 0 | 0.292 | 0 | 6.1152 | 0 | 0.408 | 0.165 | 0.48 | 0 |
| Pistachio | 0 | 1.462 | 0 | 1.118 | 4.816 | 2.4596 | 0.137 | null | 22.36 |
| nuts, raw | | | | | | | | | |
| Walnut | 0 | 0.2255 | 0 | 0.4725 | 0.546 | 0.294 | 0.063 | 1.134 | 0.42 |
| Almonds, | 0 | 0.1164 | 0 | 3.0753 | 0 | 21.7855 | 0.967 | 0 | 0 |
| unroasted | | | | | | | | | |
| Rye bread | 0 | 0.225 | 0 | 11.415 | 1.2 | 0.99 | 1.005 | 3.6 | 0 |
| Flaxseed | 0 | 0.094 | 0 | 0.616 | 0.12 | 0.062 | 0.032 | 0.86 | 0 |
| Mackerel, | 18.2 | 1 | 6 | 5.75 | 4 | 1.725 | 0.425 | 0.25 | 97.5 |
| raw | | | | | | | | | |
| UL (x/day) | 100 | 100 | ND | 35 | 2000 | 1000 | ND | ND | 3000 |
| (adults) | | | | | | | | | |

| foods | Zn | K | Fe | Cu | Se | Ca | Mg | Na |
|---|---|---|---|---|---|---|---|---|
| Chickpeas, | 1.26 | 252 | 2.14 | 0.506 | 6.2 | 90 | 52 | 492 |
| mature | | | | | | | | |
| seeds, | | | | | | | | |
| canned, | | | | | | | | |
| drained | | | | | | | | |
| solids | | | | | | | | |
| Barley, | 4.018 | 455.7 | 6.51 | 0.514 | 42.14 | 53.9 | 107.8 | 14.7 |
| pearled, | | | | | | | | |
| cooked | | | | | | | | |
| Brown rice | 1.704 | 206.4 | 1.34 | 0.254 | 13.92 | 7.2 | 93.6 | 484.8 |
| Pistachio | 1.892 | 881.5 | 3.37 | 1.118 | 6.02 | 90.3 | 104.06 | 0.86 |
| nuts, raw | | | | | | | | |
| Walnut | 1.2978 | 185.22 | 1.22 | 0.6661 | 2.05 | 41.16 | 66.36 | 0.84 |
| Almonds, | 2.652 | 623.05 | 3.15 | 0.8763 | 3.48 | 228.65 | 229.5 | 0.85 |
| unroasted | | | | | | | | |
| Rye bread | 3.42 | 498 | 8.49 | 0.558 | 92.7 | 219 | 120 | 1809 |
| Flaxseed | 0.868 | 162.6 | 1.14 | 0.244 | 5.08 | 51 | 78.4 | 6 |
| Mackerel, | 1.225 | 1115 | 1.1 | 0.137 | 91.2 | 27.5 | 82.5 | 147.5 |
| raw | | | | | | | | |
| UL (x/day) | 40 | x | 45 | 10000 | 400 | 2500 | 350 | 2300 |
| (adults) | | | | | | | | |

Reference: Institute of Medicine (US) Committee to Review Dietary Reference Intakes for Vitamin D and Calcium; Ross AC, Taylor CL, Yaktine AL, et al., editors. Washington (DC): National Academies Press (US); 2011.

### Example 7

### Estimation of Intake Quantity for Microbiome Healthy Foods

An individualized microbiome-healthy menu plan was built with the emphasis on key microbiome health indicators, namely alpha diversity, short chain fatty acids and the abundance of butyrate producing bacteria. The menu plan was built based on microbiota-healthy nutrition rules. The range of intake quantity was established for each selected food items or nutrients based on the nutrition intake data collected in 187 countries from 1990 to 2010 (Imamura et al. Lancet Glob Health. 2015 March; 3(3):e132-42).

TABLE 6
Ranges and subranges dosage range for each microbiome-friendly rule

| | Quintiles of intake | | | | | | | | | |
|---|---|---|---|---|---|---|---|---|---|---|
| | 1st | | 2nd | | 3rd | | 4th | | 5th | |
| Wholegrains (g/day) | 12 | (1.0-18) | 24 | (19-31) | 40 | (31-56) | 70 | (56-89) | 157 | (89-477) |
| Nuts and seeds (g/day) | 1.5 | (0.1-2.3) | 3.1 | (2.3-4.0) | 5.1 | (4.0-6.8) | 9.5 | (6.8-12.5) | 19.4 | (12.5-192) |
| Dietary fiber (g/day) | 14 | (7-16) | 18 | (16-19) | 21 | (19-22) | 24 | (22-26) | 28 | (26-41) |
| Sea food omega 3 FA | 22 | (3.7-40) | 56 | (40-70) | 95 | (70-141) | 215 | (141-322) | 533 | (322-5202) |
| (mg/day) | | | | | | | | | | |
| Bean and legumes (g/day) | 1.6 | (0.1-7.1) | 14 | (7.1-20) | 27 | (20-35) | 57 | (35-97) | 147 | (97-472) |

Rule Group 1: Wholegrains Consumption at 35 g/day

According to Table 5, 35 g/day of wholegrain belongs to the quintile 3. The range from wholegrain intake was set from the lowest limit of the quintile 3 of 31 g/day to the highest limit of the quintile 5 of 477 g/day. (Reference: Vanegas et al. Am J Clin Nutr. 2017 March; 105(3):635-650)

Dose range for wholegrains: 31 to 477 g/day

Rule Group 2: Beans and Legumes—Chickpea at 200 g/day

In food groups, chickpea belongs to bean and legumes. Two hundred grams per day of chickpea consumption is in the 5th quintile of the bean and legume intake (Table 5). In order to define a range, we extended the lower limit to the low end of intake in the 4th quintile. The range for chickpea was from 35 to 472 g/day. (Reference: Fernando et al. Benef Microbes. 2010 June; 1(2):197-20)7.

Dose range for chickpea: 35-472 g/day

Rule Group 3: Fiber—Brown Rice, Barley and Rye Bread

Fibers are among the most important dietary ingredients for the gut microbiota and consumption of brown rice, barley or rye bread enhances the microbiota health indicators. Fiber intake was used use to calculate the amount of food.

16 g/day of fiber in the rye bread intervention or 18.7 g/day of fiber in barley+brown rice intervention gave a significant result for microbiota health indicators. This quantity of fiber intakes was in the 2nd quintile of the global intake range (Table 5). the lower limit of fiber intake was set at 16 g/d of fiber intake. However, the highest fiber intake listed in Table 5 (41 g/day) was not high enough because the more than 50 g/day can be achieved by the menu plan algorithm. To set the upper limit for fiber intake, we referred to the fiber intake of Nigerian students who reported to consume 54.2±13.7 g/day by male and 40.5±8.5 g/day be female volunteers. We added 3 standard deviations on top of mean, which covers 99% of fiber intake in Nigerian population, and it resulted in 95.3 g/day of fiber consumption. Therefore, the upper limit of fiber intake for the microbiome menu plan is 95 g/day. Meal planning engine incorporated brown rice, barley or rye bread in daily meals to reach the quantity shown in the literature. (References: Martínez et al. ISME J. 2013 Feb; 7(2):269-80; Prykhodko et al. Front Nutr. 2018 May 29; 5:45; Adegoke et al., Br J Nutr. 2014 Jun. 28; 111(12):2146-52).

Dose range for dietary fiber: 16-95 g/day

Rule Group 4: Nuts and Seeds

Among the microbiota friendly ingredients: pistachio, almond, walnut, and flaxseed belong to the group of nuts and seeds. The quantity that were tested in a clinical study ranged from 21 g/day (0.3 g/kg for flaxseed in 70 kg man) to 86 g/day for pistachio. These amounts of intake were within the range of the 4th and 5th quintiles. The range was defined from the low end of the quintile to the high end of 5th quintile.

(References: Holscher et al. J Nutr. 2018 Jun. 1; 148(6):861-867; Lagkouvardos et al. Mol Nutr Food Res. 2015 August; 59(8):1614-28; Watson et al. Gut. 2018 November; 67(11):1974-1983).

Dose range for pistachio, almond, walnut and flaxseed: 6.8-192 g/day

Rule Group 5: Omega-3 Fatty Acids: DHA and EPA

Supplementation of DHA and EPA at 2 grams each significantly increases the abundance of Roseburia species. However, sourcing DHA and EPA only from diet is challenging and 2 grams of EPA intake, or 6.67 g of omega-3 fatty acid, exceeds the upper limit of the intake habitual consumption. Therefore, we followed a different approach to set up upper and lower limits of DHA and EPA. We set the upper limits for dietary DHA and EPA consumption according to the doses mentioned clinical study. As for the lower limit, we referenced the 5th quintile for omega-3 fatty acid consumption. Then, we converted the value to DHA, using 37% of total omega-3 fatty acid as DHA in salmon oil as a standard. This calculation gave the dose of 119 mg/day DHA as a lower limit. (References: Imamura et al., Lancet Glob Health. 2015 March; 3(3):e132-42; Dovale-Rosabal et al. Molecules, 2019 May; 24(9): 1642).

Dose range for DHA: 119 mg to 2000 mg/day DHA; Dose range for EPA: up to 2000 mg/day; or up to about 5200 mg/day in total omega-3-fatty acids.

### Example 8

### Workflow and Optimization of Menu Plan

FIG. 10 represents an example workflow for the optimization of the microbiome-health menu plan.

For building the microbiome-healthy menu plan, there were specific rules, constraints and goals in optimizing the menu plan each day and over weeks based on the microbiome-healthy foods in order to create the recommendations.

For a specific nutrient or ingredient there was an optimal range, specified by a lower and upper limit which defined the possible range for the nutrient, as well as weighting for the lower and upper deviation from the ideal range. The rules include additional information such as units and whether the rule scaled with the amount of food.

Each goal was for a specific nutrient and includes a coefficient, used as a weight in the formula which was optimized, as well as whether the nutrient should be minimized or maximized.

Rules for a Nutrient or Ingredient:

For each rule, r, corresponding to a nutrient or ingredient:

rₗₕᵣ=desired lower bound of rule

rᵤₕᵣ=desired upper bound of rule

rₗₕᵣ₀=absolute minimum for rule

rᵤₕᵣ₀=absolute maximum for rule

rₗₛ=weight for lower bound of rule

rᵤₛ=weight for upper bound of rule

These were specified goals, g, to either maximize or minimize the amount of nutrients and/or ingredients in the overall menu plan:

g_{coeff}=coefficient for goal g g_{obj} ∈ {min, max}=whether g should be maximized or minimized

Variables:

From a database of meal items, there were pre-created meals, m, which were suitable for use in menu plans. Each meal contained one or more foods in specified amounts, as well as summary nutrition information for the entire meal.

Each meal was represented by the variable f.

A variable theta, Θ, was created for each meal which represented whether the meal will be included in the menu plan. It was required to have nutritional information for each meal for all the specified rules and goals:

θ_{f}, f ∈ meals, θ_{f} ∈ {0,1}

A tag variable, t, was introduced to indicate whether the meal was tagged for each occasion (breakfast, lunch, dinner, snacks.). These tags were used to ensure the correct number of meals each day.

m=meal occasions {breakfast, lunch, dinner, snack, etc. } t_{f,m} ∈ {0, 1} f ∈ meals, m ∈ occasions

Constraints:

For each constraint, c, there was a constraint placed upon the solution to the optimization problem.

$${m}_{l} = fewest number of meal occasion m to include \\ {m}_{u} = most number of meal occasion m to include \\ {m}_{l} \leq {c}_{m} \leq {m}_{u} , for m ∈ \{ breakfast , lunch , dinner , snack , etc . \} \\ {c}_{m} = {\sum }_{f ∈ meals} {t}_{f} {θ}_{f}$$

For each rule we create two constraints with upper and lower bounds:

c_{l,r}=constraint, rₗₕᵣ≤c_{l,r}≤∞, for r ∈ rules

c_{u,r}=constraint, 0≤c_{l,r}≤rᵤₕᵣ, for r ∈ rules

For each rule, a slack variable, s, was created to represent the deviation of the menu plan from the desired range for that nutrient:

s_{l,r}=lower end slack variable for rule r

s_{u,r}=upper end slack variable for rule r

0≤s_{l,r}≤rₗₕᵣ−rₗₕᵣ₀, for r ∈ rules

0≤s_{u,r}≤rᵤₕᵣ₀−rᵤₕᵣ, for r ∈ rules

Each constraint was the sum of the amount of each food to be included in the menu plan multiplied by the amount of that nutrient in the food, plus the corresponding slack variable:

$${c}_{l , r} = lower end constraint for rule r \\ {c}_{u , r} = upper end constraint for rule r \\ {c}_{l , r} = {s}_{l , r} + {\sum }_{f ∈ meals} {θ}_{f} \cdot {f}_{r} , r ∈ rules {c}_{u , r} = {s}_{u , r} + {\sum }_{f ∈ meals} {θ}_{f} * {f}_{r} , r ∈ rules$$

where fᵣ=amount of nutrient r in food f

Objective Term

An objective term, obj, for the goals was created by summing over each goal and each food, the amount of the food to be included in the menu plan plus the amount of the nutrient in the food multiplied by the goal coefficient, normalized by the maximum amount of the nutrient in all of the possible foods.

$$o b j = {\sum }_{g ∈ goals} {\sum }_{f ∈ meal s} {θ}_{f} + \frac{{f}_{g} \cdot {g}_{c o e f f}}{max \{ f o o {d}_{g} \}}$$

where f_{g}=amount of nutrient g in food f

The menu plan was created by minimizing the sum of the slack variables over the rules weighted by the corresponding weight of the rule, plus the objective term described above.

$${min}_{θ} = {\sum }_{r ∈ rules} {s}_{l , r} * {r}_{ls} + {\sum }_{r ∈ rules} {s}_{u , r} * {r}_{us} + obj$$

The slack variables s measured the deviation of the resulting menu plan from the bounds for each nutrient specified in the rules. By minimizing the slack variables, the menu plan was able to respect the set rules. However, by using slack variables instead of specifying constraints allowed the menu plan greater leeway and recognized that it may not always be possible to exactly follow the rules.

The problem was solved by the optimization engine to produce the recommendations and resulting menu plans per day, week or month.

### Example 9

### Menu Plan User Application

FIG. 11 (screen shot) shows an example of individual data collected before the start of the menu plan for an individual user.

The resulting menu plans were visualized using a web application. As an illustrative example, FIGS. 12A, 12B and 12C shows a typical screenshot of a healthy US menu plan.

In FIG. 12C, the images were marked with a small “bacterium symbol” 1202 for those tagged as microbiome-healthy, as they contained one of the microbiome-healthy rules. The “arrows symbol” 1204 allowed the user to interchange the recipes or dishes created automatically by the engine. The meal nutritional score, marked with a symbol, “My Menu IQ” 1206, was displayed to give the meal nutritional score out of 100 for each meal occasion.

1-2 (canceled)

3. A computer implemented method comprising determining microbiome health in an individual by measurement of the improvement or maintenance of at least one of the following: (i) alpha diversity of microbial species in the intestine, (ii) butyrate production by butyrate producing bacteria or (iii) short chain fatty acid production, before and after administration of the microbiome-healthy recommendations.

4. A computer implemented method according to claim 3 wherein said method provides personalized microbiome-healthy recommendations based on individual parameters selected from the group comprising: age, gender, height, weight, BMI medical condition, and physical activity level.

5. A computer implemented method according to claim 3 wherein said method provides individualized microbiome-healthy recommendations based on dietary preferences or constraints selected from the group comprising: omnivorous diet, gluten-free, lactose-free, mediterranean, vegan, and vegetarian.

6. A computer implemented method according to claim 3 wherein said method provides recommendations for food or nutrients selected from the group consisting of:
(i) wholegrain foods;
(ii) beans and legumes;
(iii) fiber;
(iv) nuts and seeds; and
(v) omega-3 fatty acids.

7. A computer implemented method according to claim 6 wherein said method provides recommendations for food or nutrients selected from the group consisting of:
(i) wholegrain foods in the total amount of about 31 to 477 g/day;
(ii) beans and legumes in the total amount of about 35 to 472 g/day
(iii) fiber in the total amount of about 16 to 95 g/day;
(iv) nuts and seeds in the total amount of about 6 to 192 g/day; and
(v) omega-3 fatty acids in the total amount of up to about 5200 mg/day.

8. A computer implemented method according to claim 6 wherein said method provides recommendations in the category of beans and legumes wherein chickpeas in the total amount of 35 to 472 g/day are selected.

9. A computer implemented method according to claim 6 wherein fiber is selected from the group consisting of: brown rice, barley and rye in the total amount of 16 to 95 g/day.

10. A computer implemented method according to claim 6 wherein nuts and seeds are selected from the group consisting of: pistachio, almond, walnut and flaxseed in the total amount of 6 to 192 g/day.

11. A computer implemented method according to claim 6 wherein the omega-3 fatty acids are selected from the group consisting of: DHA and EPA supplements each in the amount of about 119 to 2000 mg/day; or the equivalent amount of DHA and EPA in 200 to 300 g fatty fish; or up to about 5200 mg/day in total omega-3-fatty acids.

12. A computer-implemented system for microbiome-healthy recommendations comprising:
a menu database module;
a recipe database module;
a dietary constraints module;
a nutrient scoring module; and
an optimization module
wherein said system provides microbiome-healthy recommendations to improve or maintain microbiome health in an individual.

13. A computer-implemented system for microbiome-healthy recommendations according to claim 12 wherein said system provides recommendations selected from the group comprising food recommendations, menu recommendations and recipe recommendations to improve or maintain microbiome health in an individual.

14. A computer-implemented system for microbiome-healthy recommendations according to claim 12 wherein said system provides individualized microbiome healthy recommendations based on individual user attributes selected from the group comprising: age, gender, height, weight, BMI, medical condition, physical activity level and total recommended daily energy allowance.

15. A computer-implemented system for microbiome healthy recommendations according to claim 12 wherein said system provides individualized microbiome healthy recommendations based on determination of the improvement or maintenance of at least one of the following: (i) alpha diversity of microbial species in the intestine, (ii) butyrate production or (iii) short chain fatty acid production.

16. A computer-implemented system for microbiome healthy recommendations according to claim 12 wherein said system provides individualized microbiome healthy recommendations based on dietary preferences selected from the group comprising: omnivorous diet, gluten-free, lactose-free, mediterranean, vegan, and vegetarian.

17. A computer-implemented system for microbiome healthy recommendations according to claim 12 wherein said system provides individualized microbiome-healthy recommendations for food or nutrient groups selected from the group consisting of:
(i) wholegrain foods;
(ii) beans and legumes;
(iii) fiber;
(iv) nuts and seeds; and
(v) omega-3 fatty acids.

18. A computer-implemented system for microbiome healthy recommendations according to claim 12 wherein said system provides microbiome-healthy recommendations for menus classified as suitable for breakfast, lunch, dinner or snacks.

19. (canceled)

20. A computer-implemented system for microbiome healthy recommendations according to claim 12 wherein said system provides recommendations for food or nutrient groups selected from the group consisting of:
(i) wholegrain foods in the total amount of about 31 to 477 g/day;
(ii) beans and legumes in the total amount of about 35 to 472 g/day
(iii) fiber in the total amount of about 16 to 95 g/day;
(iv) nuts and seeds in the total amount of about 6 to 192 g/day; and
(v) omega-3 fatty acids in the total amount of up to about 5200 mg/day.

21-23. (canceled)

24. A computer-implemented system for microbiome healthy recommendations according to claim 12 wherein said system provides recommendations for food or nutrient groups wherein the omega-3 fatty acids are selected from the group consisting of: DHA and EPA supplements each in the amount of about 119 to 2000 mg/day; or the equivalent amount of DHA and EPA in 200 to 300 g fatty fish; or up to about 5200 mg/day in total omega-3-fatty acids.

25. A computer-implemented system for microbiome healthy recommendations according to claim 12 wherein said system provides menus recommendations wherein the menus are tagged as microbiome-healthy for the user.

26. A computer-implemented system for microbiome healthy recommendations according to claim 12 wherein said system provides recipe recommendations wherein the recipes are tagged as microbiome-healthy for the user.
